(19) United States
(12) Patent Application Publication
(10) Pub. No.: US 2004/0241856 A1
(43) Pub. Date: Dec. 2, 2004

(54) Methods and compositions for modulating stem cells

(75) Inventor: Michael P. Cooke, Del Mar, CA (US)

Correspondence Address: GENOMICS INSTITUTE OF THE, NOVARTIS RESEARCH FOUNDATION, 10675 JOHN JAY HOPKINS DRIVE, SUITE E225, SAN DIEGO, CA, 92121-1127, US

(73) Assignee: IRM LLC, a Delaware Limited Liability Company, Hamilton (BM)

(21) Appl. No.: 10/777,028

(22) Filed: Feb. 11, 2004

Related U.S. Application Data
(60) Provisional application No. 60/447,030, filed on Feb. 12, 2003.

(51) Int. Cl. C12N 5/08; C12N 15/86

(52) U.S. Cl. 435/456; 435/372

(57) ABSTRACT

The invention provides methods for inhibiting stem cell differentiation and for increasing the effective dose of stem cells in a subject. HSC differentiation can be inhibited by applying an HSC differentiation-inhibiting polypeptide identified in the present invention to an HSC culture in vitro, or administering the polypeptide to a subject in vivo. Some other methods of the invention comprise first obtaining a population of hematopoietic stem cells, introducing into the cells an HSC differentiation-inhibiting polynucleotide disclosed herein, and expressing the HSC differentiation-inhibiting polynucleotide in the cells. Such genetically modified stem cells can be administered to a subject whereby effective dose of the stem cells in the subject can be increased. This invention further provides novel molecular markers of hematopoietic stem cells, and methods for enriching hematopoietic stem cells using these novel markers.

### CROSS-REFERENCE TO RELATED APPLICATIONS

This application claims the benefit of priority to U.S. Provisional Patent Application Ser. No. 60/447,030 (filed Feb. 12, 2003), the disclosure of which is incorporated herein by reference in its entirety and for all purposes.

### FIELD OF THE INVENTION

The present invention generally relates to methods for enriching stem cell population and for modulating stem cell differentiation, as well as to therapeutic applications of such methods. More particularly, the invention pertains to genes differentially expressed in hematopoietic stem cells and to methods of using these genes to modulate stem cell differentiation.

### BACKGROUND OF THE INVENTION

Hematopoiesis (hemopoiesis) is a process whereby multi-potent stem cells give rise to lineage-restricted progeny. The molecular basis of hematopoiesis remains poorly understood. Hematopoietic stem cells (HSCs) are the only cells in the hematopoietic system that produce other stem cells and give rise to the entire range of blood and immune system cells. These cells are able to self-proliferate, so as to maintain a continuous source of regenerative cells. When subject to particular environments and/or factors, they can differentiate to dedicated progenitor cells, where the dedicated progenitor cells may serve as the ancestor cell to a limited number of blood cell types.

HSCs and their progenies at the various development stages all play an important role in the normal function of the mammalian immune system. HSCs are of prominent therapeutic importance in many circumstances. In many diseased states, the disease is a result of some defect in the maturation process. In other situations, such as transplantation, there is a need to prevent the immune system from rejecting the transplant by irradiating the host. In neoplasia, a patient may be irradiated and/or treated with chemotherapeutic agents to destroy the neoplastic tissue, which often also damage or destroy the host immune system. Further, other situations such as a severe insult to the immune system also result in a substantial reduction in stem cells and injury to the immune system. In all these situations, it will frequently be desirable to restore stem cells to the host. For example, HSCs are the active component in bone marrow transplantation (BMT), and transplant of highly purified HSC will completely restore the hematopoietic system in a manner indistinguishable from unfractioned bone marrow.

Despite decades of research, there are currently no satisfactory methods to expand the numbers of HSCs or accurately enumerate the numbers of expanded and engraftable HSCs cells following in vitro culture. There is a need in the art for better methods for isolating, enriching, and enumerating transplantable HSCs. The instant invention fulfills this and other needs.

### SUMMARY OF THE INVENTION

In one aspect, the invention provides methods for inhibiting differentiation of mammalian stem cells. The methods entail (a) providing a population of stem cells, (b) introducing a vector comprising an HSC differentiation-inhibiting polynucleotide of the present invention into the stem cells, and (c) expressing a polypeptide encoded by the polynucleotide by culturing the modified stem cells, thereby inhibiting differentiation of the stem cells. In some of the methods, the stem cells are isolated from bone marrow. In some preferred methods, the stem cells are human hematopoietic stem cells. The human stem cells can be first selected for expression of CD38 and Thy prior to introduction of the vector. In some of the methods, the HSC differentiation-inhibiting polynucleotide encodes GATA-binding protein 3 or ID3.

In a related aspect, the invention provides methods for increasing the effective dose of hematopoietic stem cells in a mammalian subject. The methods require (a) providing a population of hematopoietic stem cells, (b) introducing into the cells an HSC differentiation-inhibiting polynucleotide of the present invention, and c) administering the genetically modified cells that express an HSC differentiation-inhibiting polypeptide to a mammalian subject; thereby increasing the effective dose of hematopoietic stem cells in the subject. In some of these methods, the administered stem cells are a subpopulation of the modified cells that are selected for expression of the polypeptide prior to administering to the subject. In some preferred methods, the subject is human, and the hematopoietic stem cells are human hematopoietic stem cells. In these methods, the hematopoietic stem cells can be selected for expression of CD34 and Thy prior to introducing into the cells the HSC differentiation-inhibiting polynucleotide.

In another related aspect, the present invention provides methods for inhibiting hematopoietic stem cell differentiation using an HSC differentiation-inhibiting polypeptide identified by the present inventor. The methods entail contacting a population of HSCs with an effective amount of the HSC differentiation-inhibiting polypeptide which inhibits differentiation of the HSCs. In some of the methods, the HSCs are present in an in vitro cell culture. In some other methods, the HSCs are present in a subject grafted with the HSCs. In some preferred methods, the subject is human.

In another aspect, the invention provides methods for isolating a population of cells that are enriched for hematopoietic stem cells (HSCs). These methods comprise (a) obtaining a sample of cells containing hematopoietic stem cells, (b) selecting cells from the sample based on expression or lack of expression of at least one known HSC surface marker, and at least one novel HSC molecule marker identified in the present invention, and (c) separating cells with the known HSC marker and at least one of the novel molecule markers; thereby isolating a population of human cells enriched for hematopoietic stem cells.

Preferably, the hematopoietic stem cells enriched with these methods are human HSCs. In some methods, the known human HSC marker is CD34+ and Thy+. In some of the methods, the at least one novel HSC marker is a human HSC surface molecule identified in the present invention.

In another aspect, the invention provides methods for enumerating hematopoietic stem cells in a population of cells. The methods entail (a) contacting the population of cells with an antibody that specifically binds to one novel HSC surface marker identified in the present invention under conditions that allow the antibody to specifically bind to the HSC surface marker, and (b) quantifying the cells recognized by the antibody; thereby enumerating hematopoietic stem cells in the population of cells. In some of these methods, the hematopoietic stem cells are human HSCs, and the population of cells are first selected for expression of CD34 and Thy prior to the contacting.

A further understanding of the nature and advantages of the present invention may be realized by reference to the remaining portions of the specification and claims.

### BRIEF DESCRIPTION OF THE DRAWINGS

FIG. 1 shows schematic structure of expression vectors for overexpressing various HSC differentiation-inhibiting genes.

FIG. 2 shows that ID3 over-expression increases the number of colony forming cells in CFC assay.

FIG. 3 shows upregulated expression of various transcription factors in mouse HSCs.

### DETAILED DESCRIPTION

I. Overview The present invention is predicated in part on the discovery by the present inventor that a number of genes are differentially expressed in hematopoietic stem cell populations (see Examples below). It was also found that some of these HSC genes slow down HSC differentiation or enhance HSC activities when they are overexpressed in HSCs. These genes are therefore termed HSC differentiation-inhibiting genes.

Using HSCs enriched from blood of normal human donors, it was found that sequences upregulated in the human HSCs include genes encoding hormones, enzymes, histone, transcription factors, secreted proteins, surface markers, and other molecules. Table 1 lists examples of these genes that are upregulated in human HSCs (CD4+Thy+) as compared to non stem cells (CD4+Thy−). Further, using HSCs isolated from two different sources, bone marrow and peripheral blood, the present inventor identified a set of genes that are differentially expressed in HSCs from both sources. Some of these genes are shown in Table 2.

Similarly, in a mouse HSC population (CD34−CD38+), a number of genes encoding proteins with diverse biochemical and cellular functions were also upregulated, including genes encoding surface antigens, transcription factors or growth factors (see Tables 3 and 4). These novel HSC genes are enriched in HSCs compared to their differentiated progeny (e.g., CD34+ CD38+ progenitor cells) or CD34+CD38− facilitator cells.

Without being bound in theory, the molecules upregulated in HSCs could play various functions in modulating HSC growth and differentiation, as well as regulating activities and functions of progenitor cells that differentiated from the HSCs. For example, increased levels of some of the surface receptors, growth factors, and secreted proteins shown in Table 2 could act in synergy in inhibiting HSC differentiation and promoting their expansion.

In accordance with these discoveries, the present invention provides methods for modulating HSC differentiation. Inhibition of HSC differentiation allows continued growth and expansion of the HSC population, and therefore provide engraftable HSCs with increased dosage and higher potency. A number of the upregulated HSC genes identified herein (e.g., shown in Tables 1, 3, and 4) can potentially function as HSC differentiation-inhibitors. For example, polypeptides encoded by the novel HSC genes disclosed herein (e.g., the growth factors or hormones shown in Table 2) can be used to inhibit HSC differentiation in vitro (e.g., by applying to an HSC cell culture) and in vivo (e.g., by administering to a subject engrafted with bone marrow or HSCs). Differentiation inhibiting activities of these molecules were exemplified by GATA3 and ID3 as shown in the Examples below.

As indicated by the GenBank accession numbers or other identification numbers or descriptions in Tables 1, 3, and 4, sequences of the upregulated human and mouse HSC genes disclosed herein are all known in the art. Thus, as detailed below, the HSC differentiation-inhibiting polynucleotide sequences can be easily obtained commercially, from the sources disclosed in the public databases, or isolated using routine techniques of molecular biology. The encoded polypeptides can also be obtained commercially or easily produced with standard procedures of recombinant techniques.

The invention also provides methods for isolating and enriching HSCs. The currently known HSC markers are not satisfactory because they cannot accurately predict homogeneity and hematopoiesis activities of cells bearing the markers. The discovery of genes differentially expressed in HSCs provides novel molecular markers for selecting and enriching HSCs. For example, antibodies against novel surface markers disclosed in the present invention (e.g., those in Tables 2, 3, 4 and 5) can be used to isolate human and mouse HSCs from a crude population of cells (e.g., bone marrow or peripheral blood). The methods can also be directed to cell populations already enriched for one or more of the known HSCs makers (e.g., CD34+, Thy+ in human, and CD38+, c-kit+, Sca1+ in mice). Further enrichment using these novel markers can lead to more homogeneous HSCs with more potent hematopoiesis activities.

In both the autologous and allogeneic setting, the time to recover from BMT is directly related to the dose of HSCs transplanted. Even a modest 2 to 3-fold expansion of engraftable HSC would afford great benefit to patients by minimizing the duration of cytopenia when patients are most susceptible to infection. Thus, isolation and expansion of more homogeneous HSCs in vitro in accordance with the present invention would make autologous and allogeneic HSC transplantation safer and more effective.

The practice of the present invention will employ, unless otherwise indicated conventional techniques of cell biology, molecular biology, cell culture, immunology and the like which are in the skill of one in the art. These techniques are fully disclosed in the art, e.g., in Sambrook et al., “Molecular Cloning A Laboratory Manual,” Cold Springs Harbor Laboratory Press (3rd ed. 2001); Carter and Sweet, “Methods of Enzymology,” Academic Press (1997); and Harlow and Lane, “Antibodies, A Laboratory Manual,” Cold Spring Harbor Press (1998).

The following sections provide more specific guidance for making and using the compositions of the invention, and for carrying out the methods of the invention.

TABLE 1
Genes upregulated in human CD34+Thy+ HSCs from peripheral blood

| Classification | Name | Description |
|---|---|---|
| Histone | H2BFL | Homo sapiens H2B histone family, memberA |
| Histone | H2AFA | Human histone genes |
| Histone | H2A/l | Homo sapiens H2A histone family, member L |
| Histone | H1F2 | Histone 2A-like protein gene |
| Histone | H2B/h | Homo sapiens H2B histone family, member H |
| Histone | HH2A/c | Human histone H2AFC gene |
| Histone | H2AFQ | Homo sapiens H2A histone family, member Q |
| HLA | HLA-DPB1 | Human MHC class II lymphocyte antigen beta chain |
| HLA | HLA-DQB1 | Human MHC class II HLA-DR2-Dw12 mRNA DQw1-beta |
| HLA | HLA-E | Homo sapiens HLA-E gene |
| Secreted-complement | PTS | Homo sapiens 6-pyruvoyltetrahydroprotein synthase |
| Secreted-complement | HFL1 | Human factor H homologue mRNA complete cds |
| Secreted-growth | MDK | Homo sapiens midkine (neurite growth-promoting factor 2) |
| factor | | |
| Secreted-hormone | OXT | Homo sapiens oxytocin, prepro-(neurophysin 1) mRNA |
| Secreted-hormone | AVP | Homo sapiens arginine vasopressin mRNA |
| Signaling-GTP | R-Ras | Human R-ras |
| Signaling-GTP | GCHFR | Homo sapiens GTP cyclohydrolase I feedback regulatory protein |
| Signaling-GTP | GUCY1A3 | Homo sapiens guanylate cyclase 1, soluble, alpha 3 |
| Signaling-Kinase | WAF1 | Human DNA sequence from PAC 431A14WAF1 |
| Signaling-Kinase | ITPKB | Homo sapiens inositol 1,4,5-triphosphate 3-kinase B |
| Signaling-Kinase | PPKCL | Homo sapiens protein kinase C, eta |
| Signaling-Kinase | PPKCZ | Homo sapiens protein kinase C, zeta |
| Signaling-SH3 | SKAP55 | Homo sapiens src kinase-associated phosphoprotein of 55 kDa |
| Stress | PTGS2 | Homo sapiens prostaglandin-endoperoxide synthase 2 |
| Stress | CYP2A13 | Human cytochrome P450 |
| Stress | CYP2D6 | Human mRNA for cytochrome P450 dbl variant b |
| Stress-apoptosis | BCL2A1 | Homo sapiens BCL-2-related protein 1 |
| Structural | CALB1 | Homo sapiens calbindin 1 |
| Structural | Elastin | Human elastin gene |
| Structural | KRT18 | Human mRNA fragment for cytokeratin 18 |
| Surface-Ig | IGM | Human gene for immunoglobulin mu |
| Surface-Ig | VH4 | Human IgM heavy chain variable V-D-J region (VH4) gene |
| Surface-other | APP | Homo sapiens APP complete sequence |
| Surface-receptor | BDKRB1 | Human bradykinin B1 receptor |
| Surface-receptor | TLR1 | Human mRNA for KIAA0012 gene |
| Surface-receptor | 5T4 | Homo sapiens 5T4 oncofetal trophoblast glycoprotein |
| Surface-receptor | EFL-2 | Homo sapiens EHK1 receptor tyrosine kinase ligand |
| Surface-receptor | EV12A | Homo sapiens ecotropic viral integration site 2A |
| Surface-receptor | FLT3 | Homo sapiens fms-related tyrosine kinase 3 |
| Surface-receptor | TNFSF10 | Human tumor necrosis factor (ligand) superfamily, member 10 |
| Surface-receptor | LTB | Human lymphotoxin beta |
| Surface-receptor | CDW52 | Homo sapiens mRNA for CAMPATH-1 |
| Surface-receptor | CLECSF2 | Homo sapiens C-type lectin (activation-induced) |
| Surface-unknown | GliPR | Human glioma pathogenesis-related protein |
| Transport | LRP | Homo sapiens Irp mRNA |
| Transcription-RUNT | AML1 | Human AML1 protein |
| Transcription-PAR-bZIP | TEF | Human hepatic leukemia factor |
| Transcription-FKH | FKHR | Homo sapiens forkhead protein |
| Transcription- | MN1 | Homo sapiens chromosome 22q11.2 MDR region |
| suppressor | | |
| Transcription-bHLH | ID1 | Homo sapiens inhibitor of DNA binding 1 |
| Transcription-bHLH | ID3 | Homo sapiens HLH 1R21 mRNA for helix-loop-helix protein |
| Transcription-bHLH | EPAS1 | Homo sapiens endothelial PAS domain protein 1 |
| Transcription-bHLH | ID2 | Homo sapiens inhibitor of DNA binding 2 |
| Transcription-GATA | HGATA3 | Homo sapiens GATA-binding protein 3 |
| Transcription-HMG | hTcf-4 | Homo sapiens mRNA for hTCF-4 |
| Transcription-HOX | PHOX1 | Human homeobox protein |
| Transcription-HOX | MEIS1 | Homo sapiens MEIS protein |
| Transcription- | RBP-MS | Homo sapiens RNA-binding protein gene with multiple slicing |
| slicing | | |
| Transcription- | TCEA2 | Homo sapiens transcription elongation factor A |
| Translation | | |
| Unknown | DIF2 | IEX-1 = radiation-inducible immediate-early gene |
| Unknown | | Homo sapiens chromosome 17clone hRPC.906_A_24 |
| Unknown | | Homo sapiens chromosome 22q13 BAC clone CIT987SK-384D8 |
| Unknown | A-362G6.1 | Human chromosome 16 BAC clone CIT987SK-A-362G6 |
| Unknown | LST1 | Homo sapiens LST1 mRNA |
| Unknown | KIAA0125 | Homo sapiens KIAA0125 gene product |

TABLE 2
Genes Upregulated in Human HSCs from both Bone Marrow and Peripheral Blood

| Classification | Name | Description |
|---|---|---|
| Hormone | AVP | Homo sapiens arginine vasopressin mRNA |
| Hormone | | Corticotropin releasing hormone-binding protein |
| Enzyme | GUCY1A3 | Homo sapiens guanylate cyclase 1, soluble, alpha 3 |
| Enzyme | PPKCZ | Homo sapiens protein kinase C, zeta |
| Enzyme | | Iduronate 2-sulfatase (Hunter syndrome) |
| Transcription factor | HLF | Human hepatic leukemia factor |
| Transcription factor | GATA3 | Homo sapiens GATA-binding protein 3 |
| Transcription | Evil | Homo sapiens ecotropic viral integration site 1 |
| Transcription | PMX1 | Paired mesoderm homeo box 1 |
| Transcription | MN1 | Meningioma (disrupted in balanced translocation) |
| Secreted protein | | Tetranectin (plasminogen-binding protein) |
| Secreted protein | | H factor (complement)-like 1 |
| Surface molecule | | Transient receptor potential channel 1 |
| Surface molecule | DLK1 | Delta-like homolog (Drosophila) |
| Surface molecule | EphA3 | Ephrin-A3 |
| Surface molecule | TNFSF10 | Human tumor necrosis factor (ligand) superfamily, member 10 |
| Surface molecule | | Interferon induced transmembrane protein |
| Surface molecule | | Ecotropic viral integration site 2A |
| Surface molecule | | Sortilin-related receptor, L(DLR class) A rep |
| Surface molecule | | Major histocompatibility complex, class I, E |
| Surface molecule | | KIAA0125 gene product |

II. Definition

Unless defined otherwise, all technical and scientific terms used herein have the same meaning as commonly understood by those of ordinary skill in the art to which this invention pertains. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY (1991). In addition, the following definitions are provided to assist the reader in the practice of the invention.

The term “analog” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved traits (such as higher binding affinity for a target molecule) is an approach that is well known in pharmaceutical chemistry.

As used herein, “contacting” has its normal meaning and refers to combining two or more agents (e.g., polypeptides or small molecule compounds) or combining agents and cells (e.g., a polypeptide and a cell). Contacting can occur in vitro, e.g., combining two or more agents or combining a test agent and a cell or a cell lysate in a test tube or other container. Contacting can also occur in a cell or in situ, e.g., contacting two polypeptides in a cell by coexpression in the cell of recombinant polynucleotides encoding the two polypeptides, or in a cell lysate.

An “effective amount or dose” is an amount sufficient to effect beneficial or desired results. An effective amount may be administrated in one or more administrations. Determination of an effective amount is within the capability of those skilled in the art. Particularly preferred subjects of the invention in general include living mammals such as human, mice and rabbit, most preferred are humans. The administration of an HSC differentiation-inhibiting polypeptide, or a genetically modified cell comprising a polynucleotide sequence of the invention, may be by conventional means, for example, injection, oral administration, inhalation and others. Appropriate carries and diluents may be included in the administration of the polypeptide or the modified cells. Samples including the modified cells and progeny thereof may be taken and tested to determine transduction efficiency.

The term “fragment” when used in connection with an amino acid sequence means a part of a reference sequence and having at least 10 amino acid residues, preferably 50 amino acids residues, even more preferably 100 amino acid residues and most preferably 200 amino acid residues which are substantially identical to the reference amino acid sequences. Where referring to a nucleotide sequence, the term means a nucleotide sequence including part of the reference sequence and comprising as few as at least 30, 50, 75, 80, 100 or more contiguous nucleotides, preferably at least 200, 300, 400, 500, 600, or more contiguous nucleotides, even more preferably at least 800, 1000, 1500, 2000 or more contiguous nucleotides that are identical to the reference sequence.

The term “functional equivalent” when referring to a polypeptide means a protein having a like function and like or improved specific activity, and a similar amino acid sequence. In some embodiments, a functionally equivalent is a variant in which one or more amino acid residues are substituted with conserved or non-conserved amino acid residues, or one in which one or more amino acid residues includes a substituent group. Conservative substitutions are the replacements, one for another, among the aliphatic amino acids Ala, Val, Leu and Ile; interchange of the hydroxl residues Ser and Thr; exchange of the acidic residues Asp and Glu; substitution between amide residues Asn and Gln; exchange of the basic residues Lys and Arg; and replacements among aromatic residues Phe and Tyr.

A “heterologous sequence” or a “heterologous nucleic acid,” as used herein, is one that originates from a source foreign to the particular host cell, or, if from the same source, is modified from its original form. Thus, a heterologous gene in a host cell includes a gene that, although being endogenous to the particular host cell, has been modified. Modification of the heterologous sequence can occur, e.g., by treating the DNA with a restriction enzyme to generate a DNA fragment that is capable of being operably linked to the promoter. Techniques such as site-directed mutagenesis are also useful for modifying a heterologous nucleic acid.

The term “homologous” when referring to proteins and/or protein sequences indicates that they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity is routinely used to establish homology. Higher levels of sequence similarity, e.g., 30%, 40%, 50%, 60%, 70%, 80%, 90%, 95% or 99% or more can also be used to establish homology. Methods for determining sequence similarity percentages, e.g., BLASTP and BLASTN using default parameters, are well known and described in the art.

The terms “identical sequence” and “sequence identity” in the context of two nucleic acid sequences or amino acid sequences refer to the residues in the two sequences which are the same when aligned for maximum correspondence over a specified comparison window. A “comparison window”, as used herein, refers to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman (1981) Adv. Appl. Math. 2:482; by the alignment algorithm of Needleman and Wunsch (1970) J. Mol. Biol. 48:443; by the search for similarity method of Pearson and Lipman (1988) Proc. Nat. Acad. Sci U.S.A. 85:2444; by computerized implementations of these algorithms (including, but not limited to CLUSTAL in the PC/Gene program by Intelligentics, Mountain View, Calif.; and GAP, BESTFIT, BLAST, FASTA, or TFASTA in the Wisconsin Genetics Software Package, Genetics Computer Group (GCG), 575 Science Dr., Madison, Wis., U.S.A.). The CLUSTAL program is well described by Higgins and Sharp (1988) Gene 73:237-244; Higgins and Sharp (1989) CABIOS 5:151-153; Corpet et al. (1988) Nucleic Acids Res. 16:10881-10890; Huang et al (1992) Computer Applications in the Biosciences 8:155-165; and Pearson et al. (1994) Methods in Molecular Biology 24:307-331. Alignment is also often performed by inspection and manual alignment.

The term “isolated” means that the material is removed from its original environment (e.g., the natural environment if it is naturally occurring). For example, a naturally-occurring nucleic acid, polypeptide, or cell present in a living animal is not isolated, but the same polynucleotide, polypeptide, or cell separated from some or all of the coexisting materials in the natural system, is isolated, even if subsequently reintroduced into the natural system. Such nucleic acids can be part of a vector and/or such nucleic acids or polypeptides could be part of a composition, and still be isolated in that such vector or composition is not part of its natural environment. When referring to a cell population, it means that homogeneous cells expressing a given set of molecular markers constitute at least 60%, preferably 75%, more preferably 90%, and most preferably 95% of the total number of cells in the population.

The terms “substantially identical” nucleic acid or amino acid sequences means that a nucleic acid or amino acid sequence comprises a sequence that has at least 90% sequence identity or more, preferably at least 95%, more preferably at least 98% and most preferably at least 99%, compared to a reference sequence using the programs described above (preferably BLAST) using standard parameters. For example, the BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)). Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

The terms “nucleic acid” and “polynucleotide” refer to a deoxyribonucleotide or ribonucleotide polymer in either single- or double-stranded form, and unless otherwise limited, encompasses known analogues of natural nucleotides that hybridize to nucleic acids in manner similar to naturally occurring nucleotides. A “polynucleotide sequence” is a nucleic acid (which is a polymer of nucleotides (A,C,T,U,G, etc. or naturally occurring or artificial nucleotide analogues) or a character string representing a nucleic acid, depending on context. Either the given nucleic acid or the complementary nucleic acid can be determined from any specified polynucleotide sequence.

The term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance. A polylinker provides a convenient location for inserting coding sequences so the genes are operably linked to the promoter. Polylinkers are polynucleotide sequences that comprise a series of three or more closely spaced restriction endonuclease recognition sequences.

As used herein the term “overexpression” refers to expression of a polypeptide brought about by genetic modification of a host cell with a nucleic acid sequence encoding the polypeptide. Overexpression may take place in cells normally lacking expression of the polypeptide (e.g., an HSC differentiation-inhibiting polypeptide). It can also occur in cells with endogenous expression of the polypeptide. While overexpression may take place in any cell type, preferred host cells for overexpressing an HSC differentiation-inhibiting polypeptide are hematopoietic stem cells.

The terms “polypeptide” and “protein” are used interchangeably herein, and refer to a polymer of amino acid residues, e.g., as typically found in proteins in nature. A “mature protein” is a protein which is full-length and which, optionally, includes glycosylation or other modifications typical for the protein in a given cell membrane.

A “variant” of a molecule such as an HSC differentiation-inhibiting polypeptide is meant to refer to a molecule substantially similar in structure and biological activity to either the entire molecule, or to a fragment thereof. Thus, provided that two molecules possess a similar activity, they are considered variants as that term is used herein even if the composition or secondary, tertiary, or quaternary structure of one of the molecules is not identical to that found in the other, or if the sequence of amino acid residues is not identical. In some embodiments, a variant differs in amino acid sequence from a reference polypeptide by one or more substitutions, additions, deletions, truncations which may be present in any combination. Among preferred variants are those that vary from a reference polypeptide by conservative amino acid substitutions. Such substitutions are those that substitute a given amino acid by another amino acid of like characters. The following non-limiting list of amino acids are considered conservative replacements: a) alanine, serine, and threonine; b) glutamic acid and asparatic acid; c) asparagine and glutamine d) arginine and lysine; e) isoleucine, leucine, methionine and valine and f) phenylalaine, tyrosine and tryptophan. Most highly preferred are variants that retain the same biological function and activity as the reference polypeptide from which it varies.

III. Promoting HSC Expansion by Inhibiting Differentiation

In addition to novel markers and methods for isolating HSCs, the invention also provides methods for inhibiting or blocking differentiation of mammalian hematopoietic stem cells, thereby promoting expansion of the stem cells. A number of the novel HSC marker genes identified in the present invention can inhibit or block HSC differentiation. Examples of such differentiation-inhibiting genes are shown in Tables 1 and 2 (for human HSC) and Tables 3 and 4 (for mouse HSC). For example, as described in the Examples below, human stem cells overexpressing GATA-binding protein 3 slows differentiation of the cells. HSCs overexpressing ID3 increased colony forming cells, indicating enhanced HSC activity as compared to a control. These differentiation-inhibiting molecules can be used in the present invention to inhibit HSC differentiation and thereby promoting expansion in vitro. They can also be used in vivo to increase the effective dose of engrafted HSCs in a subject.

The term HSC differentiation-inhibiting molecules (polynucleotides and the encoded polypeptides) include the molecules shown in Tables 1-4 that inhibit or slow HSC differentiation. Polynucleotides with substantial sequence identity are also encompassed. In addition, they also include variants, analogs, fragments, or functional derivatives of the HSC differentiation-inhibiting molecules shown in Tables 1-4. These differentiation-inhibiting molecules can be obtained from any species. Preferably, they are from mammalian species including human, mouse, and chicken. The HSC differentiation-inhibiting molecules can also be from any source whether natural, synthetic or recombinant.

Differentiation is defined as the restriction of the potential of a cell to self-renew and is normally associated with a change in the functional capacity of the cell. The term “inhibiting” or “blocking” differentiation is used broadly in the context of this invention and includes not only the prevention of differentiation but also encompasses altering or slowing differentiation process of a cell. Differentiation of a stem cell can be determined by methods well known in the art and these include analysis for surface markers associated with cells of a defined differentiated state.

An HSC differentiation-inhibiting polypeptide of the present invention encodes an HSC differentiation-inhibiting polypeptide that blocks or slows down differentiation of the HSC cells (e.g., as listed in Tables 1-4). As shown in the Tables, these molecules include hormones, secreted proteins, or growth factors. These molecules also include transcription factors. One or more of these HSC differentiation-inhibiting polypeptides, or fragments thereof, can be applied to HSC cells in vitro, e.g., in a cell culture. These cells can be cultured and grown as described herein or other methods well known in the art. The appropriate amount of these differentiation-inhibiting polypeptides to be used in the cultures can be easily determined in accordance with stem cell culturing procedures described herein or knowledge well known in the art. By culturing the HSC in the presence of these molecules, differentiation of the cells can be inhibited or slowed, resulting in enhanced growth of engraftable HSCs.

In addition to promoting HSC expansion in vitro, the HSC differentiation-inhibiting polypeptides of the invention can also be administered directly to a subject to promote in vivo growth of HSCs. For example, a subject engrafted with bone marrow or a population of HSCs can also be administered an effective amount of an HSC differentiation-inhibiting polypeptide or fragment thereof (e.g., the secreted proteins or growth factors shown in Table 1 and Tables 3-4). The polypeptide can be administered to the subject prior to, concurrently with, or subsequent to transplantation of the bone marrow or HSCs. Preferably, the polypeptide and the HSCs are administered to the subject simultaneously.

Other than using a differentiation-inhibiting polypeptide, inhibition of HSC differentiation can also be achieved using an HSC differentiation-inhibiting polynucleotide to genetically modify HSCs. HSC differentiation-inhibiting polynucleotides suitable for these methods include some of the genes upregulated in HSCs (as shown in Tables 1 and 3). They encode HSC differentiation-inhibiting polypeptides that block or slow down differentiation of the HSC cells. Some of these methods require first isolation of a population of hematopoietic cells, e.g., a population of CD34⁺Thy⁺ human cells or CD34⁻CD38⁺ mouse cells as described above, from a source of such cells. An HSC differentiation-inhibiting polynucleotide of the invention can then be introduced into the cells whereby the cells are genetically modified.

Once the cells are genetically modified, they are cultured in the presence of at least one cytokine in an amount sufficient to support growth of the modified cells. The modified cells are then selected wherein the encoded polypeptide is overexpressed and differentiation is blocked. The genetically modified cells thus obtained may be used immediately (e.g., in transplant), cultured and expanded in vitro, or stored for later uses. The modified HSCs may be stored by methods well known in the art, e.g., frozen in liquid nitrogen.

Genetic modification as used herein encompasses any genetic modification method of introduction of an exogenous or foreign gene into mammalian cells (particularly human stem cell and hematopoietic cells). The term includes but is not limited to transduction (viral mediated transfer of host DNA from a host or donor to a recipient, either in vitro or in vivo), transfection (transformation of cells with isolated viral DNA genomes), liposome mediated transfer, electroporation, calcium phosphate transfection or coprecipitation and others. Methods of transduction include direct co-culture of cells with producer cells (Bregni et al., Blood 80:1418-1422, 1992) or culturing with viral supernatant alone with or without appropriate growth factors and polycations (Xu et al., Exp. Hemat. 22:223-230, 1994).

Various in vitro and in vivo assays are well known in the art for the measurement of the functional compositions of hematopoietic cell populations. See, e.g., Quesenberry et al. eds., Stem Cell Biology and Gene Therapy, Wiley-Liss Inc. 1998—Chapter 5, Hematopoietic Stem cells: Proliferation, Purification and Clinical Applications, pgs 133-160. Other examples of suitable assays are also known in the art. For example, the long term culture-initiating cell (LTCIC) assay involves culturing a cell population on stromal cell monolayers for approximately 5 weeks and then testing in a 2 week semisolid media culture for the frequency of clonogenic cells retained (Sutherland et al., Blood 74:1563 (1989)). The Colony Forming Cells (CFC) assay or Colony-Forming Unit Culture (CFUC) assay involves use of cell count as the number of colony-forming units per unit volume or area of a sample. The assay is used to measure clonal growth of quickly maturing progenitors in semi-solid media supplemented with serum and growth factors. Depending on the growth factors used to stimulate growth mature and/or primitive progenitors may be determined. Cobblestone area forming colony (CAFC) assays measure clonal proliferation of long-lived progenitors supported by stromal cell monolayers and growth factor/serum supplemented media. On the appropriate stromal monolayers, cells pluripotent for myeloid and lymphoid lineages may be determined. (Young et al., Blood 88:1619, 1996). SCID-hu bone assays measure the proliferation and multilineage differentiation of cells with bone marrow repopulating activity. These cells are likely to contribute to durable engraftment in clinical transplantation. SCID-hu thymus assays measure the proliferation and differentiation in thymocytes. Both bone marrow repopulating and more mature T-lineage progenitors may be measured.

A polynucleotide encoding an HSC differentiation-inhibiting molecule is typically introduced to a host cell in a vector. The vector typically includes the necessary elements for the transcription and translation of the inserted coding sequence. Methods used to construct such vectors are well known in the art. For example, techniques for constructing suitable expression vectors are described in detail in Sambrook et al., Molecular Cloning: A Laboratory Manual, Cold Spring Harbor Press, N.Y. (3^{rd} Ed., 2000); and Ausubel et al., Current Protocols in Molecular Biology, John Wiley & Sons, Inc., New York (1999).

Vectors may include but are not limited to viral vectors, such as baculovirus, retroviruses, adenoviruses, adeno-associated viruses, and herpes simplex viruses; bacteriophages; cosmids; plasmid vectors; synthetic vectors; and other recombination vehicles typically used in the art. Vectors containing both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). Specific examples include, pSG, pSV2CAT, pXtl from Stratagene; and pMSG, pSVL, pBPV and pSVK3 from Pharmacia.

Preferred vectors include retroviral vectors (see, Coffin et al., “Retroviruses”, Chapter 9 pp; 437-473, Cold Springs Harbor Laboratory Press, 1997). Vectors useful in the invention can be produced recombinantly by procedures well known in the art. For example, WO94/29438, WO97/21824 and WO97/21825 describe the construction of retroviral packaging plasmids and packing cell lines. Exemplary vectors include the pCMV mammalian expression vectors, such as pCMV6b and pCMV6c (Chiron Corp.), pSFFV-Neo, and pBluescript-Sk+. Non-limiting examples of useful retroviral vectors are those derived from murine, avian or primate retroviruses. Common retroviral vectors include those based on the Moloney murine leukemia virus (MoMLV-vector). Other MoMLV derived vectors include, Lmily, LINGFER, MINGFR and MINT (Chang et al., Blood 92:1-11, 1998). Additional vectors include those based on Gibbon ape leukemia virus (GALV) and Moloney murine sacroma virus (MOMSV) and spleen focus forming virus (SFFV). Vectors derived from the murine stem cell virus (MESV) include MESV-MiLy (Agarwal et al., J. of Virology, 72:3720-3728, 1998). Retroviral vectors also include vectors based on lentiviruses, and non-limiting examples include vectors based on human immunodeficiency virus (HIV-1 and HIV-2).

In producing retroviral vector constructs, the viral gag, pol and env sequences can be removed from the virus, creating room for insertion of foreign DNA sequences. Genes encoded by foreign DNA are usually expressed under the control a strong viral promoter in the long terminal repeat (LTR). Selection of appropriate control regulatory sequences is dependent on the host cell used and selection is within the skill of one in the art. Numerous promoters are known in addition to the promoter of the LTR. Non-limiting examples include the phage lambda PL promoter, the human cytomegalovirus (CMV) immediate early promoter; the U3 region promoter of the Moloney Murine Sarcoma Virus (MMSV), Rous Sacroma Virus (RSV), or Spleen Focus Forming Virus (SFFV); Granzyme A promoter; Granzyme B promoter, CD34 promoter; and the CD8 promoter. Additionally inducible or multiple control elements may be used.

Such a construct can be packed into viral particles efficiently if the gag, pol and env functions are provided in trans by a packing cell line. Therefore, when the vector construct is introduced into the packaging cell, the gag-pol and env proteins produced by the cell, assemble with the vector RNA to produce infectious virons that are secreted into the culture medium. The virus thus produced can infect and integrate into the DNA of the target cell, but does not produce infectious viral particles since it is lacking essential packaging sequences. Most of the packing cell lines currently in use have been transfected with separate plasmids, each containing one of the necessary coding sequences, so that multiple recombination events are necessary before a replication competent virus can be produced. Alternatively the packaging cell line harbors a provirus. The provirus has been crippled so that although it may produce all the proteins required to assemble infectious viruses, its own RNA cannot be packaged into virus. RNA produced from the recombinant virus is packaged instead. Therefore, the virus stock released from the packaging cells contains only recombinant virus. Non-limiting examples of retroviral packaging lines include PA12, PA317, PE501, PG13, PSI.CRIP, RDI 14, GP7C-tTA-G10, ProPak-A (PPA-6), and PT67. Reference is made to Miller et al., Mol. Cell Biol. 6:2895, 1986; Miller et al., Biotechniques 7:980, 1989; Danos et al., Proc. Natl. Acad. Sci. USA 85:6460, 1988; Pear et al., Proc. Natl. Acad. Sci. USA 90:8392-8396, 1993; and Finer et al., Blood 83:43-50, 1994.

Other suitable vectors include adenoviral vectors (see, Frey et al., Blood 91:2781, 1998; and WO 95/27071) and adeno-associated viral vectors. These vectors are all well know in the art, e.g., as described in Chatterjee et al., Current Topics in Microbiol. And Immunol., 218:61-73, 1996; Stem cell Biology and Gene Therapy, eds. Quesenberry et al., John Wiley & Sons, 1998; and U.S. Pat. Nos. 5,693,531 and 5,691,176. The use of adenovirus-derived vectors may be advantageous under certain situation because they are not capable of infecting non-dividing cells. Unlike retroviral DNA, the adenoviral DNA is not integrated into the genome of the target cell. Further, the capacity to carry foreign DNA is much larger in adenoviral vectors than retroviral vectors. The adeno-associated viral vectors are another useful delivery system. The DNA of this virus may be integrated into non-dividing cells, and a number of polynucleotides have been successful introduced into different cell types using adeno-associated viral vectors.

In some embodiments, the construct or vector will include two or more heterologous polynucleotide sequences; a) the nucleic acid sequence encoding an HSC differentiation-inhibiting polypeptide of the invention, and b) one or more additional nucleic acid sequence. Preferably the additional nucleic acid sequence is a polynucleotide which encodes a selective marker, a structural gene, a therapeutic gene, a ribozyme, or an antisense sequence.

A selective marker may be included in the construct or vector for the purposes of monitoring successful genetic modification and for selection of cells into which DNA has been integrated. Non-limiting examples include drug resistance markers, such as G148 or hygromycin. Additionally negative selection may be used, for example wherein the marker is the HSV-tk gene. This gene will make the cells sensitive to agents such as acyclovir and gancyclovir. Selection may also be made by using a cell surface marker, for example, to select overexpression of an HSC differentiation-inhibiting polypeptide by fluorescence activated cell sorting (FACS). The NeOR (neomycin/G148 resistance) gene is commonly used but any convenient marker gene may be used whose gene sequences are not already present in the target cell can be used. Further non-limiting examples include low-affinity Nerve Growth Factor (NGFR), enhanced fluorescent green protein (EFGP), dihydrofolate reductase gene (DHFR) the bacterial hisD gene, murine CD24 (HSA), murine CD8a(lyt), bacterial genes which confer resistance to puromycin or phleomycin, and beta.-glactosidase.

The additional polynucleotide sequence(s) may be introduced into the host cell on the same vector as the polynucleotide sequence encoding the polypeptides of the invention or the additional polynucleotide sequence may be introduced into the host cells on a second vector. In a preferred embodiment, a selective marker will be included on the same vector as the HSC differentiation-inhibiting polynucleotide.

Typically, the host cells for expressing the HSC differentiation-inhibiting polynucleotide are mammalian stem cells, e.g., HSCs from humans, mice, monkeys, farm animals, sport animals, pets, and other laboratory rodents and animals. These cells can be obtained, cultured, and manipulated as described above and in Potten C. S. ed., Stem Cells, Academic Press, 1997; Stem Cell Biology and Gene Therapy, eds. Quesenberry et al., John Wiley & Sons Inc., 1998; and Gage et al., Ann. Rev. Neurosci. 18:159-192, 1995.

IV. Novel Molecular Markers for Isolating and Enriching HSCs

As detailed in the Examples below, the present inventor identified a number of genes that are differentially expressed in human and mouse HSCs. These genes, which can play a role in regulating hematopoiesis as well as activities of HSCs and progenitor cells, are suitable as markers for selecting and enriching HSCs from diverse populations of cells. As exemplified in Tables 1-4, these HSC markers include transmembrane proteins (e.g., receptors), growth factor, transcription factors, as well as other proteins with diverse cellular and biochemical functions.

Employing these novel HSC markers, the present invention provides methods for isolating stem cells from any vertebrate, particularly mammalian, species. In general, one or more of the novel markers can be targeted in the methods. Selection with these markers can be performed alone with a crude population of cells (e.g., bone marrow). The selection scheme can also be used in combination with other selection and purification procedures, e.g., to further select HSCs from cells already enriched for other known HSC surface markers.

In some embodiments, the novel markers for selecting and enriching HSCs are cell surface markers. As described in the Examples, a number of the genes upregulated in the human and mouse HSCs encode transmembrane proteins (see also Tables 2 and 7). These proteins provide novel surface markers for isolating HSCs from or enumerating HSCs in a population of diverse cells (e.g., bone marrow). These methods are useful for isolating stem cells from primates, e.g. human, monkeys, gorillas, domestic animals, bovine, equine, ovine, porcine, and etc. Isolation of HSCs bearing these novel markers can be performed with the same procedures disclosed herein for the other phenotypic markers.

In some embodiments, selection of the novel HSC markers utilizes antibodies that recognize the novel HSC markers. This includes preparing an antibody to a novel HSC marker (e.g., a surface marker) of the invention and purifying the antibody. By exposing a population of hematopoietic cells or crude cells to the antibody and allowing the exposed cells to bind with the antibody, cells bearing the novel HSC marker can be isolated. Techniques including antibody preparation and purification are well known and routinely practiced in the art. See, e.g., Harlow and Lane, Antibodies: A Laboratory Manual, Cold Spring Harbor Press (1998). Such antibodies encompass any antibody or fragment thereof either native or recombinant, synthetic or naturally derived, which retains sufficient specificity to bind specifically to an HSC marker. They may be monoclonal or polyclonal, and can be produced using the novel HSC marker protein or a fragment or variant thereof. In addition, antibodies that recognize some of these marker proteins may also be obtained commercially.

When combined with other selection procedures, the particular order by which hematopoietic cells are separated from other cells is not critical to this invention. When a genetically modified HSC cell is to be selected (as detailed above), the specific cell types may be separated either prior to genetic modification or after genetic modification. In some methods, crude cell samples are initially separated by markers indicating unwanted cells, then with a negative selection, followed by separations for markers or marker levels indicating that the cells belong to the stem cell population, and finally positive selection with novel markers of the present invention. In some other methods, following the initial crude separation, the cells can be directly subject to enrichment for at least one of the novel HSC markers.

For example, an initial crude cell population can be first purified to remove major cell families from the bone marrow or other hematopoietic cell source. A negative selection can then be carried out by targeting some of the cell surface antigens (e.g., Lin, CD34 for mouse HSCs). A further positive selection can be performed to isolate a cell population with specific stem cell markers (e.g., CD34 and Thy for human HSC, and c-kit, Sca-1, or CD38 for mouse HSC). Thereafter, additional selections can be carried out using one or more of the novel HSC surface markers disclosed herein.

The starting cell populations for selecting and enriching HSC can be obtained from bone marrow or other hematopoietic source. Stem cells and progenitor cells from bone marrow constitute only a small percentage (e.g., about 0.01 to about 0.1%) of the bone marrow cells. Bone marrow cells may be obtained from a source of bone marrow, e.g. tibiae, femora, spine, fetal liver, and other bone cavities. Other sources of hematopoietic stem cells include embryonic yolk sac, fetal live, fetal and adult spleen, and blood including adult peripheral blood and umbilical cord blood (To et al., Blood 89:2233-2258, 1997).

Procedures for isolation of bone marrow are well known in the art. For example, an appropriate solution may be used to flush the bone. For example, the solution can be a balanced salt solution conveniently supplemented with fetal calf serum or other naturally occurring factors. These components can be present in conjunction with an acceptable buffer at low concentration, generally from about 5 to 25 mM. Convenient buffers include but are not limited to HEPES, phosphate and lactate buffers. Bone marrow can also be aspirated from the bone in accordance with other conventional techniques well known in the art.

As indicated above, to isolate the HSC cells, a relatively crude separation can be initially used to remove major cell families from the bone marrow or other hematopoietic cell source. Various techniques may be employed to separate the cells to initially remove cells of dedicated lineage. These include physical separation, magnetic separation using antibody-coated magnetic beads, affinity chromatography, and cytotoxic agents joined to a monoclonal antibody or used in conjunction with a monoclonal antibody. Also included is the use of fluorescence activated cell sorters (FACS) wherein the cells can be separated on the basis of the level of staining of the particular antigens. These techniques are well known to those of ordinary skill in the art and are described in various references including U.S. Pat. Nos. 5,061,620; 5,409,8213; 5,677,136; and 5,750,397; and Yau et al., Exp. Hematol. 18:219-222, 1990).

Monoclonal antibodies are particularly useful for this initial separation procedure. The antibodies may be attached to a solid support to allow for separation. In some methods, magnetic bead separations are used to attach the antibodies. Conjugating the antibodies with markers such as magnetic beads, e.g., using biotin-avidin link, allows for direct separation of bound cells from the unbound cells. Antibodies (e.g., monoclonal antibodies) directed to the various surface markers of these differentiated cells can be obtained commercially or prepared using methods routinely practiced in the art.

To select HSCs, this initial separation allows removal of large numbers of cells of the hematopoietic system of various lineages, such as thymocytes, T-cells, pre-B cells, B-cells, granulocytes, myelomonocytic cells, and platelets. Cells that can be separated in this stage also include other minor cell populations, e.g., megakaryocytes, mast cells, eosinophils and basophils. Generally, at least about 70%, usually 80% or more of the total hematopoietic cells will be removed. Since there will be positive selection at the later selection steps, it is not essential to remove at the initial stage every dedicated cell class, such as the minor population members, the platelets, and erythrocytes. However, it is preferable that there be positive selection for all of the cell lineages, so that in the final positive selection the number of dedicated cells present is minimized.

Phenotypes of surface antigen of the dedicated lineage cells are known in the art. For example, CD34 is expressed on most immature T-cells also called thymocytes, and these cells lack cell surface expression of CD1, CD2, CD3, CD4, and CD8 antigens. CD45RA is a useful T-cell marker. The best known T-cell marker is the T-cell receptor (TCR). There are presently two defined types of TCRs, TCR-2 (consisting of α and β polypeptides) and TCR-1 (consisting of δ and γ polypeptides). B cells may be selected, for example, by expression of CD19 and CD20. Myeloid cells may be selected, for example, by expression of CD14, CD15, and CD16. NK cells may be selected based on expression of CD56 and CD16. Erythrocytes may be identified by expression of glycophorin A. Compositions enriched for progenitor cells capable of differentiation into myeloid cells, dendritic cells, or lymphoid cells also include the phenotypes CD45RA⁺ CD34⁺ Thy1⁺ and CD45RA⁺ CD10⁺ Lin⁻CD34⁺. Other useful markers for various cell types are also known in the art.

The separation techniques employed should maximize the retention of viability of the fraction to be collected. For the initial separations, various techniques of differing efficacy may be employed. The particular technique employed will depend upon efficiency of separation, cytotoxicity of the methodology, ease and speed of performance, and necessity for sophisticated equipment and/or technical skill. Procedures for separation may include magnetic separation, using antibody-coated magnetic beads, affinity chromatography, cytotoxic agents joined to a monoclonal antibody or used in conjunction with a monoclonal antibody, e.g. complement and cytotoxins, and “panning” with antibody attached to a solid matrix, e.g. plate. Techniques providing accurate separation include fluorescence activated cell sorters, which can have varying degrees of sophistication, e.g. a plurality of color channels, low angle and obtuse light scattering detecting channels, and impedance channels.

Following the initial coarse selection, positive and/or negative selection using various other known stem cell markers as well as the novel HSC markers disclosed herein can be followed. In some methods, human HSCs are isolated using markers such as CD34⁺ and Thy⁺ as discussed in the Examples below. In some methods, human HSCs are selected for a phenotype of CD34⁺ Thy1⁺ Lin⁻. Other examples of enriched phenotypes include: CD2⁻, CD3⁻, CD4⁻, CD8⁻, CD10⁻, CD14⁻, CD15⁻, CD19⁻, CD20⁻, CD33⁻, CD34⁻, CD38^{lo/-}, CD45RA⁻, CD 59^{+/-}, CD71⁻, CDW109⁺, glycophorin⁻, AC133⁺, HLA⁻DR^{+/-}, c-kit⁺, and EM⁺. Lin⁻ refers to a cell population selected on the basis of lack of expression of at least one lineage specific marker, for example CD2, CD3, CD14, and CD56. The combination of expression markers used to isolate and define an enriched HSC population may vary depending on various factors and may vary as other expression markers become available.

Similarly, mouse HSCs can be selected for one or more of the known markers such as Lin⁻, c-kit⁺, Sca-1⁺, CD38⁺, and CD34⁻ (see Example 3). In other methods, murine HSCs with similar properties to the human CD34⁺ Thy-1⁺ Lin⁻ may be identified by kit+Thy-1.1^{lo} Lin^{-/lo} Sca-1⁺ (KTLS). Other phenotypes are well known, e.g., as described in U.S. Pat. No. 6,451,558. When CD34 expression is combined with selection for Thy-1, a composition comprising approximately fewer than 5% lineage committed cells can be isolated (U.S. Pat. No. 5,061,620).

Once the cells are harvested and optionally separated, the cells are cultured in a suitable medium comprising a combination of growth factors that are sufficient to maintain growth. The term culturing refers to the propagation of cells on or in media of various kinds. It is understood that the descendants of a cell grown in culture may not be completely identical (either morphologically, genetically or phenotypically) to the parent cell. Methods for culturing stem cells and hematopoietic cells are well known to those skilled in the art. Any suitable culture container may be used, and these are readily available from commercial vendors. The seeding level is not critical, and it will depend on the type of cells used. In general, the seeding level will be at least 10 cells per ml, more usually at least about 100 cells per ml and generally not more than 10⁶ cells per ml.

Various culture media can be used and non-limiting examples include Iscove's modified Dulbecco's medium (IMDM), X-vivo 15 and RPMI-1640. These are commercially available from various vendors. The formulations may be supplemented with a variety of different nutrients, growth factors, such as cytokines and the like. In general, the term cytokine refers to any one of the numerous factors that exert a variety of effects on cells, such as inducing growth and proliferation. The cytokines may be human in origin or may be derived from other species when active on the cells of interest. Included within the scope of the definition are molecules having similar biological activity to wild type or purified cytokines, for example produced by recombinant means, and molecules which bind to a cytokine factor receptor and which elicit a similar cellular response as the native cytokine factor.

The medium can be serum free or supplemented with suitable amounts of serum such as fetal calf serum, autologous serum or plasma. If cells or cellular products are to be used in humans, the medium will preferably be serum free or supplemented with autologous serum or plasma (see, e.g., Lansdorp et al., J. Exp. Med. 175:1501, 1992; and Petzer et al., PNAS 93:1470, 1996).

Examples of compounds that can be used to supplement the culture medium are thrombopoietin (TPO), Flt3 ligand (FL), c-kit ligand (KL, also known as stem cell factor, SCF, or Stl), Interleukin (e.g., IL-1, IL-2, IL-3, IL-6, soluble IL-6 receptor, IL-11, and IL-12), granulocyte-colony stimulating factor (G-CSF), granulocyte macrophage-colony stimulating factor (GM-CSF), leukemia inhibitory factor (LIF), MIP-1:c, and erythropoietin (EPO). These compounds may be used alone or in any combination. When murine stem cells are cultured, a preferred non-limiting medium includes mIL-3, mIL-6 and mSCF.

Concentration range of these compounds to be used in cultures can be determined according to knowledge well known in the art. For example, a general preferred range of TPO is from about 0.1 ng/mL to about 5000 μg/mL, more preferred is from about 1.0 ng/mL to about 1000 ng/mL, even more preferred from about 5.0 ng/mL to about 300 ng/mL. A preferred concentration range for each of FL and KL is from about 0.1 ng/mL to about 1000 ng/mL, more preferred is from about 1.0 ng/mL to about 500 ng/mL. IL-6 is a preferred factor to be included in the culture, and a preferred concentration range is from about 0.1 ng/mL to about 500 ng/mL, and more preferred from about 1.0 ng/mL to about 100 ng/mL. Hyper IL-6, a covalent complex of IL-6 and IL-6 receptor may also be used in the culture.

Other molecules can also be added to the culture media, for instance, adhesion molecules, such as fibronection or RetroNectin™ (commercially produced by Takara Shuzo Co., Otsu Shigi, Japan). Fibronectin is a glycoprotein that is found throughout the body, and its concentration is particularly high in connective tissues where it forms a complex with collagen.

V. Therapeutic Applications

HSC's are the active component in bone marrow transplantation (BMT). The use of purified HSCs transplant as opposed to bone marrow provides the advantage that transplant of harmful non-HSC cells in the bone marrow is avoided. In the autologous cancer or autoimmune setting, the use of purified HSCs minimizes the possibility of giving tumor or diseased cells back to the patient along with the bone marrow. In allogenic transplantion, using high doses of HSCs overcomes rejection by the recipient immune system. Thus, expansion of HSCs would make autologous and allogeneic HSC transplantation safer and more effective.

The present invention provides methods for inhibiting HSC differentiation and promoting HSC expansion in vivo in a subject, e.g., a human subject engrafted with HSCs. Using HSC differentiation-inhibiting molecules identified in the present invention, these methods allow expansion of non-differentiated stem cells and increase the dose of HSCs either ex vivo or in vivo, thereby potentially allowing more rapid engraftment. The HSC differentiation-inhibiting molecules can be expressed in the engrafted HSCs. It can also be separately provided to the subject receiving the HSC graft, e.g., expressed from a vector introduced into the subject. In addition, the HSC differentiation-inhibiting molecules can also be administered to the subject as an expressed polypeptide, e.g., a growth factor. As a result, differentiation of the cells is blocked or slowed down, resulting in expansion of non-differentiated stem cells.

Some methods of the invention provide ex vivo gene therapy for transplanting genetically modified HSCs cells into a subject. For example, vectors expressing an HSC differentiation-inhibiting polypeptide can be delivered to HSCs explanted from an individual subject, followed by reimplantation of the cells into a subject, usually after selection for cells that have incorporated the vector. Procedures for modifying host cells with an HSC differentiation-inhibiting polynucleotide (e.g., GATA3) are described above. In addition, ex vivo cell transfection for diagnostics, research, or for gene therapy (e.g., via re-infusion of the transfected cells into the host organism) is well known in the art. For a review of gene therapy procedures, see Anderson, Science 256: 808-813, 1992; Nabel & Felgner, TIBTECH 11: 211-217, 1993; Mitani & Caskey, TIBTECH 11: 162-166, 1993; Mulligan, Science 260: 926-932, 1993; Dillon, TIBTECH 11: 167-175, 1993; Miller, Nature 357: 455-460, 1992; Van Brunt, Biotechnology 6: 1149-1154, 1998; Vigne, Restorative Neurology and Neuroscience 8: 35-36, 1995; Kremer & Perricaudet, British Medical Bulletin 51: 31-44, 1995; Haddada et al., in Current Topics in Microbiology and Immunology (Doerfler & Böhm eds., 1995); and Yu et al., Gene Therapy 1: 13-26, 1994).

For therapeutic applications, the genetically modified HSC cells are maintained for a period of time sufficient for overexpression of HSC differentiation-inhibiting polypeptide. A suitable time period will depend inter alia upon cell type used and is readily determined by one skilled in the art. In general, genetically modified cells of the invention may overexpress HSC differentiation-inhibiting polypeptide for the lifetime of the host cell. Preferably, for hematopoietic cells the time period will be in the range of 1 to 45 days, more preferably in the range of 1 to 30 days, even more preferably in the range of 1 to 20 days, still more preferably in the range of 1 to 10 days, and most preferably in the range of 1 to 5 days.

Other than ex vivo gene therapy, vectors expressing an HSC differentiation-inhibiting polypeptide can also be delivered in vivo. This is carried out by administering to an individual subject the expression vector, typically by systemic administration (e.g., intravenous, intraperitoneal, intramuscular, subdermal, or intracranial infusion) or topical application. Methods for in vivo gene therapy are also well known in the art, e.g., as described in the literatures noted above.

As described above, other than gene therapy, therapeutic expansion of HSCs in a subject can also be achieved by directly applying an HSC differentiation-inhibiting polypeptide (or its fragment or functional derivative) to a subject. The subject can be simultaneously engrafted with HSCs. The subject can also be one that has not been subject to HSC transplant. Typically, in such applications, the HSC differentiation-inhibiting polypeptide (e.g., GATA3) is administered to the subject in a pharmaceutical composition. The pharmaceutical compositions typically comprise at least one active ingredient together with one or more acceptable carriers thereof. Suitable carriers for preparing the pharmaceutical compositions, appropriate dosages, and suitable routes of administration of the compositions can all be readily determined by following methods well known in the art. See, e.g., Gilman et al., eds., Goodman and Gilman's: The Pharmacological Bases of Therapeutics, 8th ed., Pergamon Press, 1990; Remington: The Science and Practice of Pharmacy, Mack Publishing Co., 20^{th} ed., 2000; Avis et al., eds., Pharmaceutical Dosage Forms: Parenteral Medications, published by Marcel Dekker, Inc., N.Y., 1993; and Lieberman et al., eds., Pharmaceutical Dosage Forms: Tablets, published by Marcel Dekker, Inc., N.Y., 1990.

### EXAMPLES

The following examples are provided to illustrate, but not to limit the present invention.

### Example 1

### Genes Upregulated in Human HSCs

This Example describes RNA profiling of human hematopoietic stem cells and characterization of genes upregulated in the HSCs. All procedures and assays employed herein to study the human HSCs have been described in the art, e.g., as noted above.

CD34⁺ cells were first isolated from blood of six normal human donors using magnetic beads. Flow activated cell sorting (FACS) was then used to purify CD34⁺Thy⁺ (stem enriched) and CD34⁺Thy⁻ (stem depleted) cell populations. The two populations of cells (total 12 samples, 6 CD34⁺Thy⁺ and 6 CD34⁺Thy⁻) were assayed for bioactivity with the CFC assay. RNA profiling (Thy⁺ vs Thy⁻) was then carried out to identify genes differentially expressed in stem cells. Results of the profiling are shown in Table 1. The data indicate that the upregulated genes encode proteins with diverse biochemical and cellular functions.

In addition, genes upregulated in CD34⁺Thy⁺ HSCs from two different sources, bone marrow and peripheral blood, were compared for overlapping sequences that are enriched in HSCs from both sources. A total of 30 genes were found to have been upregulated in HSCs from both sources. An exemplary list of these genes is shown in Table 2. Both HSC types contain transcription factors some of which are known proto-oncogenes (e.g., GATA3, HLF, Evi1, PMX1, MN1, ATF3).

Further, the results indicate that HSCs from peripheral blood, but not HSCs from bone marrow, are enriched in histones and inhibitory HLH transcription factors (ID1, ID2, and ID3). The data also suggest new cell surface markers for HSCs. Examples include 5T4, EphA3, TNFSF3, EVI2b, DLK1. Several potential neuropeptides are also upregulated, including Vasopression (AVP), Oxytocin (OXT), and Vasodilators.

### Example 2

### Inhition of HSC Differentiation by Overexpressing an HSC Differentiation-Inhibiting Polypeptide

The Example describes effects on HSC differentiation by constitutive expression of an HSC differentiation-inhibiting gene in CD34+Thy+ cells using retroviral vectors. First, effect of overexpressing ID3 was analyzed with colony-forming cell (CFC) assay. Other assays such as cobblestone area forming cell (CAFC) assay and NOD/SCID (nonobese diabetic mice with severe combined immunodeficiency disease) repopulating cell assay can also be used in these analyses. These assays can be performed as described as described above and are well known in the art (e.g., Kusadasi et al., Leukemia 14: 1944-53, 2000; and Larochelle et al., Nature Medicine, 2: 1329-1337, 1996).

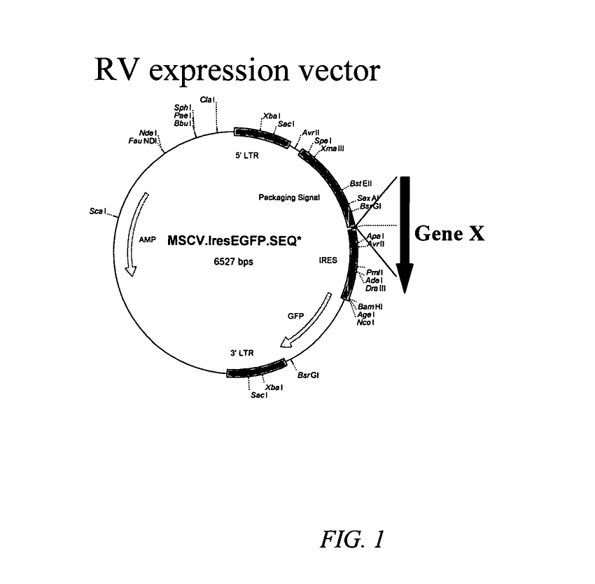

FIG. 1 illustrates the schematic structure of the retroviral vectors used in the study. Gene X in the figure denotes any of these HSC genes (e.g., ID3) to be examined. The vectors also express the green-fluorescence protein (GFP). When the GFP gene is transfected into or infected cells, the encoded GFP shines green under ultraviolet light and thus enables the detection of the transfected or infected cell in a simple manner.

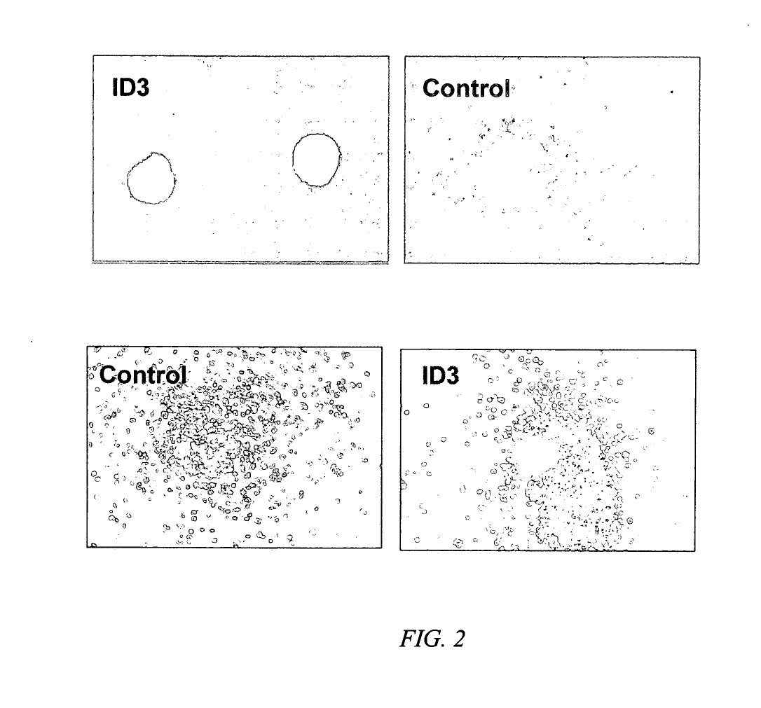

A vector harboring the HSC gene (e.g., ID3 or GATA3) was transfected into the CD34⁺ cells. Cells expressing the gene were sorted and assayed with the CFC assay. As shown in FIG. 2, ID3 over-expression increased the number of colony forming cells (e.g., primitive BFU-E colonies). This suggests enhanced HSC activity, indicating that differentiation of the stem cells has been slowed down.

The HSC differentiation-inhibiting genes were also examined for their effects on HSC growth in liquid culture. The effect of GATA3 over-expression on human HSC differentiation was examined in liquid culture. Here, stem cells were transfected with the same vectors described above (which harbor the ID1 gene, GATA3 gene, or no HSC gene), and grown in liquid culture. CD34⁺ and GFP⁺ cells were sorted. Expression of CD34 was monitored during the culture. Cells without transfection were used in a control analysis. The results indicate that, as compared to the control, ID1 had no effect on differentiation of the CD34⁺ cells. However, expression of GATA3 significantly slowed the differentiation process as indicated by the rate of reduction of CD4⁺ cells.

### Example 3

### Novel Molecular Markers Expressed in Mouse HSCs

This Example describes use of RNA expression profiling to characterize purified mouse HSCs. Mouse HSCs were purified using a combination of antibodies to cell-surface markers. The following three cell populations were purified from murine bone marrow as described in Zhao et al., Blood 96: 3016-22, 2000; and Zhong et al., Blood 100: 3521-6, 2002.

| Cell type | Immunophenotype | HSC activity |
|---|---|---|
| LT-HSC | Lin⁻, c-kit⁺, Sca-1⁺, CD38⁺, CD34⁻ | 1× |
| Facilitator | Lin⁻, c-kit⁺, Sca-1⁺, CD38⁻, CD34⁺ | 0.1× |
| Cells | | |
| Progenitor | Lin⁻, c-kit⁺, Sca-1⁺, CD38⁺, CD34⁺ | 0.1× |
| Cells | | |

Cells were purified from normal BL6 mice using flow cytometry. Three different preparations of sorted cells for each population were prepared and combined prior to the isolation of total RNA. The RNA was quantified using the Ribogreen fluorescence-based solution assay (e.g., as described in Jones et al., Anal Biochem 265: 368-74, 1998). 10 ng of each pooled RNA preparation was labeled in duplicates using the triple labeling procedure (as described, e.g., in Hrabovszky et al., J. Histochem. Cytochem. 43: 363-370, 1995) and hybridized to affymetrix U74A gene chips according to the manufacturer's instructions. Intensity values were obtained for each gene and sample using GeneChip software. These Average difference (AD) values were exported to a spreadsheet program and analyzed by first filtering for genes which are expressed above a threshold criteria (50 in at least two samples), and whose average for each population was expressed >2× or <2× between any two cell populations and where ANOVA analysis showed a significant difference (P<0.01) between any two populations.

Examples of genes upregulated in HSCs are shown in Table 3. The genes were analyzed for patterns using Genespring software and arranged by functional gene classification using GO ontogeny. Accession numbers or identification numbers from other public databases of these genes, as well as levels of up-regulation of these genes in HSCs as compared to non-HSCs, are also shown in the table.

### Example 4

### Characterization of Genes Differentially Expressed in Mouse HSCs

To correlate stem cell activity of the three subsets with gene expression, a hypothetical stem cell activity pattern corresponding to the in vivo repopulating activity of the three subsets was generated and used for comparison of the normalized expression levels of each differentially expressed gene identified above. Principle Component Analysis (PCA) on the stem cell expression data was performed to identify gene expression patterns. This is an unsupervised computational method used to identify major patterns in diverse data types including gene expression data (Alter et al., Proc Natl Acad Sci USA 97:10101-10106, 2000; and Holter et al., Proc Natl Acad Sci USA 97:8409-8414, 2000). The correlation analysis of the gene expression patterns of the differentially expressed genes with stem cell activity identified genes with highly significant (Pearson R>0.95) correlations. These genes are shown in Table 4. In addition to genes upregulated in HSCs, the analysis also identified genes whose expression negatively correlated with LTR HSCs (i.e., down-regulated expression). Examples of these genes are shown in Table 5.

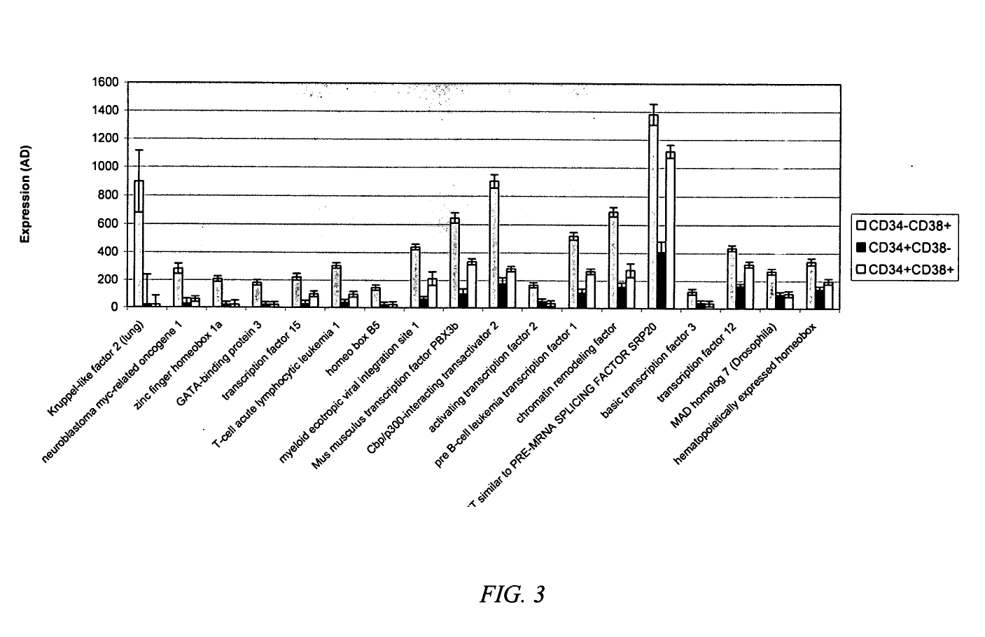

Some of the differentially expressed genes were further analyzed and classified according to their biological functions. The results are shown in Table 6. As shown in Tables 3, 4, and 6, the upregulated genes in mouse HSCs also encode proteins of diverse biological properties, similar to genes upregulated in the human HSCs. For example, a number of transmembrane proteins were enriched in the mouse HSCs, as exemplified in Table 7. These molecules can be useful as novel surface markers for isolating HSCs. Some of transcription factors that are upregulated in the mouse HSCs are shown in Table 8. Their upregulated expression levels in the CD34⁻CD38⁺ HSCs relative to that in the facilitator cells (CD38⁻CD34⁺) and progenitor cells (CD34⁺CD38⁺) are shown in FIG. 3.

The expression of several known transcription regulation factors was found to correlate positively with LTR HSC activity. These include Cited2, GATA3, Hdac3, Irf6, Jun B, Nmyc1, Rnps1, Xbp1, and Zfp292. Little is known regarding the role of these specific transcription factors in the control of HSC biology. These essential transcription factors could play an important role in regulating HSC development and differentiation.

To determine if any of the differentially expressed transcription factors are themselves regulating transcription in LTR HSCs, we performed a search of putative upstream regulatory regions (10 kb upstream of start codons) of the interrogated genes for binding sites of the nine transcription factors. Statistical analysis of these results revealed that only the binding sites of GATA were significantly enriched (P<0.05) within the differentially expressed genes. Interestingly, this list contains a large fraction (20 of 52) of the genes whose expression positively correlated with HSC activity, suggesting the possibility that Gata may play an important role in the control of LTR HSC biology. A small number of gene (3 of 20) whose expression is negatively correlated with HSC activity also contained Gata binding sites, suggesting the possibility that low levels of Gata expressed in STR HSC may influence gene expression at later stages.

To confirm the data from expression profiling, we performed semi-quantitative RT-PCR on total RNA extracted from the three BM subsets for three of the LTR HSC genes identified. These included the transcription factors Gata 3, Jun B, and the thrombopoietin receptor c-Mpl. The results demonstrated that all three mRNAs are expressed at significantly higher levels in CD38⁺CD34⁻ cells compared to the other two subsets.

TABLE 3
Genes Upregulated in Mouse HSCs

| | | | | HSC/ |
|---|---|---|---|---|
| Symbol | Description | RefSeq | Swiss Prot Keywords | non-HSC |
| AU044919 | expressed sequence AU044919 | AU044919 | Glycoprotein | 79.7 |
| | | | Immunoglobulin C region | |
| | | | Immunoglobulin domain | |
| Klf2 | Kruppel-like factor 2 (lung) | NM 008452 | Activator DNA-binding | 44.9 |
| | | | Metal-binding Nuclear | |
| | | | protein Repeat | |
| | | | Transcription regulation | |
| | | | Zinc-finger | |
| Car1 | carbonic anhydrase 1 | NM 009799 | Lyase Zinc | 36.8 |
| Mm.220154 | Mus musculus anti-HIV-1 reverse | NA | None | 30.1 |
| | transcriptase single-chain variable | | | |
| | fragment mRNA, complete cds | | | |
| 2010309G21Rik | RIKEN cDNA 2010309G21 gene | none | Immunoglobulin C region | 28.8 |
| | | | Immunoglobulin domain | |
| NA | M80423:Mus castaneus IgK chain | M80423 | None | 20.9 |
| | gene, C-region, 3 end/cds = (0,322) | | | |
| | /gb = M80423/gi = 196865 | | | |
| | /ug = Mm.46804/len = 323 mRNA | | | |
| Fragilis | Fragilis | NM 025378 | None | 17.1 |
| Smoc1 | SPARC related modular calcium | NM 022316 | None | 15.8 |
| | binding 1 | | | |
| 5830413E08Rik | RIKEN cDNA 5830413E08 gene | NM 029083 | None | 14.9 |
| 5830431A10Rik | RIKEN cDNA 5830431A10 gene | none | None | 14.4 |
| AI325941 | expressed sequence AI325941 | AI325941 | None | 14.2 |
| Cdkn1c | cyclin-dependent kinase inhibitor 1C (P57) | NM 009876 | Alternative splicing | 14.1 |
| | | | Cell cycle | |
| Lisch7 | liver-specific bHLH-Zip transcription | none | None | 13.9 |
| | factor | | | |
| AW108012 | expressed sequence AW108012 | AW108012 | None | 13.8 |
| Akr1c13 | aldo-keto reductase family 1, member C13 | NM 013778 | None | 13.3 |
| 0910001L24Rik | RIKEN cDNA 0910001L24 gene | NM 022419 | None | 12.7 |
| AI842353 | expressed sequence AI842353 | AI842353 | None | 11.7 |
| Tgm2 | transglutaminase 2, C polypeptide | NM 009373 | Acyltransferase Calcium- | 11.4 |
| | | | binding Transferase | |
| Nckap1 | NCK-associated protein 1 | none | Transmembrane | 11.3 |
| Serpina3g | serine (or cysteine) proteinase | none | None | 11.3 |
| | inhibitor, clade A, member 3G | | | |
| 1700008C22Rik | RIKEN cDNA 1700008C22 gene | none | None | 10.4 |
| Nmyc1 | neuroblastoma myc-related oncogene 1 | NM 008709 | DNA-binding Nuclear | 10.4 |
| | | | protein Phosphorylation | |
| | | | Proto-oncogene | |
| Zfhx1a | zinc finger homeobox 1a | NM 011546 | Activator DNA-binding | 10.4 |
| | | | Homeobox Metal-binding | |
| | | | Nuclear protein Repeat | |
| | | | Repressor Transcription | |
| | | | regulation Zinc-finger | |
| H2-Eb1 | histocompatibility 2, class II antigen E beta | NM 010382 | Glycoprotein MHC II | 10.0 |
| | | | Signal Transmembrane | |
| AU044919 | expressed sequence AU044919 | AU044919 | Glycoprotein | 9.9 |
| | | | Immunoglobulin C region | |
| | | | Immunoglobulin domain | |
| Gbp2 | guanylate nucleotide binding protein 2 | NM 010260 | None | 9.5 |
| Gabbr1 | gamma-aminobutyric acid (GABA-B) | NM 019439 | Alternative splicing Coiled | 9.5 |
| | receptor, 1 | | coil G-protein coupled | |
| | | | receptor Glycoprotein | |
| | | | Postsynaptic membrane | |
| | | | Repeat Signal | |
| | | | Transmembrane | |
| D8Ertd69e | DNA segment, Chr 8, ERATO Doi 69, | none | None | 9.2 |
| | expressed | | | |
| Gata3 | GATA binding protein 3 | NM 008091 | Activator DNA-binding | 9.1 |
| | | | Nuclear protein T-cell | |
| | | | Transcription regulation | |
| | | | Zinc-finger | |
| C130052I12Rik | RIKEN cDNA C130052I12 gene | NM 146047 | None | 8.7 |
| 0610025I19Rik | RIKEN cDNA 0610025I19 gene | NM 029555 | None | 8.6 |
| Tcf15 | transcription factor 15 | NM 009328 | None | 8.6 |
| H2-Aa | histocompatibility 2, class II antigen A, alpha | NM 010378 | 3D-structure Glycoprotein | 8.5 |
| | | | MHC II Signal | |
| | | | Transmembrane | |
| Tal1 | T-cell acute lymphocytic leukemia 1 | NM 011527 | Chromosomal | 8.3 |
| | | | translocation | |
| | | | Differentiation DNA- | |
| | | | binding Phosphorylation | |
| | | | Proto-oncogene | |
| | | | Transcription regulation | |
| Myoz1 | myozenin 1 | NM 021508 | None | 7.9 |
| 4930421J07Rik | RIKEN cDNA 4930421J07 gene | none | None | 7.4 |
| Igh-6 | immunoglobulin heavy chain 6 | none | Alternative splicing | 7.3 |
| | (heavy chain of IgM) | | Glycoprotein | |
| | | | Immunoglobulin C region | |
| | | | Immunoglobulin domain | |
| | | | Transmembrane | |
| Hoxb5 | Homeo box B5 | NM 008268 | Developmental protein | 7.3 |
| | | | DNA-binding Homeobox | |
| | | | Nuclear protein | |
| | | | Transcription regulation | |
| Col9a1 | procollagen, type IX, alpha 1 | NM 007740 | Alternative splicing | 7.2 |
| | | | Cartilage Collagen | |
| | | | Connective tissue | |
| | | | Extracellular matrix | |
| | | | Glycoprotein | |
| | | | Hydroxylation Repeat | |
| | | | Signal | |
| Meis1 | myeloid ecotropic viral integration | NM 010789 | None | 7.1 |
| | site 1 | | | |
| Ela1 | elastase 1, pancreatic | none | None | 7.0 |
| Hiat1 | hippocampus abundant gene transcript 1 | NM 008246 | None | 7.0 |
| Fah | fumarylacetoacetate hydrolase | NM 010176 | Hydrolase Phenylalanine | 6.9 |
| | | | catabolism Tyrosine | |
| | | | catabolism | |
| Cypf13 | cytochrome P450 CYP4F13 | NM 130882 | None | 6.7 |
| NA | :Mus musculus transcription factor | AF020200 | None | 6.5 |
| | PBX3b (PBX3b) mRNA, complete | | | |
| | cds/cds = (118, 1173)/gb = | | | |
| | AF020200/gi = 2432016/ug = | | | |
| | Mm.7331/len = 2467 mRNA | | | |
| Igj | immunoglobulin joining chain | NM 152839 | Glycoprotein Signal | 6.3 |
| NA | :AV336991 Mus musculus cDNA, 3 | AV336991 | None | 6.2 |
| | end/clone = 6332407A01 | | | |
| | /clone_end = 3/gb = AV336991 | | | |
| | /gi = 6377043/ug = Mm.99212 | | | |
| | /len = 201/NOTE = replacement for | | | |
| | probe set(s) 100264_f_at on MG-U74A mRNA | | | |
| Ctla2b | cytotoxic T lymphocyte-associated | none | Repeat Signal T-cell | 6.1 |
| | protein 2 beta | | | |
| Serpinb6 | serine (or cysteine) proteinase | NM 009254 | Serine protease inhibitor | 5.8 |
| | inhibitor, clade B, member 6 | | Serpin | |
| Mm.29940 | ESTs | NA | None | 5.8 |
| AU043625 | expressed sequence AU043625 | NM 133910 | None | 5.8 |
| Col4a1 | procollagen, type IV, alpha 1 | none | Basement membrane | 5.6 |
| | | | Collagen Connective tissue | |
| | | | Extracellular matrix | |
| | | | Glycoprotein | |
| | | | Hydroxylation Repeat | |
| | | | Signal | |
| Igh-4 | immunoglobulin heavy chain 4 | none | Alternative splicing | 5.5 |
| | (serum IgG1) | | Glycoprotein | |
| | | | Immunoglobulin C region | |
| | | | Immunoglobulin domain | |
| Siat6 | sialyltransferase 6 (N-acetyllacosaminide | NM 009176 | Glycoprotein | 5.4 |
| | alpha 2,3-sialyltransferase) | | Glycosyltransferase Golgi | |
| | | | stack Signal-anchor | |
| | | | Transferase | |
| | | | Transmembrane | |
| Igk-C | immunoglobulin kappa chain, constant region | none | None | 5.4 |
| Sdpr | Serum deprivation response | NM 138741 | None | 5.4 |
| Dusp1 | dual specificity phosphatase 1 | NM 013642 | Cell cycle Hydrolase | 5.3 |
| Cited2 | Cbp/p300-interacting transactivator, | NM 010828 | Alternative splicing | 5.2 |
| | with Glu/Asp-rich carboxy-terminal | | Nuclear protein | |
| | domain, 2 | | | |
| Epor | erythropoietin receptor | NM 010149 | Glycoprotein Receptor | 5.1 |
| | | | Signal Transmembrane | |
| Mm.200980 | Mus musculus, Similar to | NA | None | 5.0 |
| | translocation protein 1, clone | | | |
| | IMAGE: 5347105, mRNA, partial cds | | | |
| Atf2 | activating transcription factor 2 | none | Activator Alternative | 5.0 |
| | | | splicing DNA-binding | |
| | | | Metal-binding Nuclear | |
| | | | protein Phosphorylation | |
| | | | Transcription regulation | |
| | | | Zinc-finger | |
| Ccne1 | cyclin E1 | NM 007633 | Cell cycle Cell division | 5.0 |
| | | | Cyclin Nuclear protein | |
| | | | Phosphorylation | |
| Mllt3 | myeloid/lymphoid or mixed lineage- | NM 027326 | None | 4.9 |
| | leukemia translocation to 3 homolog | | | |
| | (Drosophila) | | | |
| D5Ertd40e | DNA segment, Chr 5, ERATO Doi 40, expressed | none | None | 4.9 |
| Zfp216 | zinc finger protein 216 | NM 009551 | None | 4.8 |
| Syp | synaptophysin | NM 009305 | Calcium-binding | 4.8 |
| | | | Glycoprotein Nerve | |
| | | | Phosphorylation Repeat | |
| | | | Synapse Synaptosome | |
| | | | Transmembrane | |
| Nedd4 | neural precursor cell expressed, | NM 010890 | Ligase Repeat Ubiquitin | 4.7 |
| | developmentally down-regulted gene 4 | | conjugation | |
| Pbx1 | pre B-cell leukemia transcription factor 1 | NM 008783 | None | 4.7 |
| 6330407G11Rik | RIKEN cDNA 6330407G11 gene | NM 023423 | None | 4.6 |
| Ash1 | absent, small, or homeotic discs 1 | NM 138679 | None | 4.5 |
| | (Drosophila) | | | |
| Lrmp | lymphoid-restricted membrane protein | NM 008511 | None | 4.5 |
| Casp8ap2 | caspase 8 associated protein 2 | NM 011997 | None | 4.5 |
| Mm.30163 | Mus musculus, clone IMAGE: 4952607, mRNA | NA | None | 4.5 |
| Ctsl | cathepsin L | NM 009984 | Glycoprotein Hydrolase | 4.5 |
| | | | Lysosome Signal Thiol | |
| | | | protease Zymogen | |
| Sfpq | splicing factor proline/glutamine rich | NM 023603 | None | 4.4 |
| | (polypyrimidine tract binding protein | | | |
| | associated) | | | |
| 2010004A03Rik | RIKEN cDNA 2010004A03 gene | none | None | 4.3 |
| Car2 | carbonic anhydrase 2 | NM 009801 | Lyase Zinc | 4.2 |
| Mm.22896 | ESTs | NA | None | 4.1 |
| AI573938 | expressed sequence AI573938 | none | None | 3.9 |
| Vasp | vasodilator-stimulated phosphoprotein | none | Actin-binding | 3.9 |
| | | | Phosphorylation | |
| AA408451 | expressed sequence AA408451 | AA408451 | None | 3.7 |
| Pftk1 | PFTAIRE protein kinase 1 | NM 011074 | None | 3.6 |
| Tieg | TGFB inducible early growth response | NM 013692 | DNA-binding Metal-binding | 3.6 |
| | | | Nuclear protein | |
| | | | Repeat Repressor | |
| | | | Transcription regulation | |
| | | | Zinc-finger | |
| Igk-V28 | immunoglobulin kappa chain variable 28 (V28) | none | Immunoglobulin C region | 3.6 |
| | | | Immunoglobulin domain | |
| Mm.1806 | Mus musculus, Similar to KIAA1404 protein, | NA | None | 3.5 |
| | clone IMAGE: 5252426, mRNA, partial cds | | | |
| Mm.25115 | ESTs | NA | None | 3.5 |
| Ccrn4l | CCR4 carbon catabolite repression 4-like | none | Biological rhythms | 3.5 |
| | (S. cerevisiae) | | | |
| Cpo | coproporphyrinogen oxidase | NM 007757 | Heine biosynthesis Iron | 3.5 |
| | | | Mitochondrion | |
| | | | Oxidoreductase Porphyrin | |
| | | | biosynthesis Transit peptide | |
| Nupr1 | nuclear protein 1 | NM 019738 | None | 3.5 |
| Mm.5510 | similar to gene overexpressed in astrocytoma | NA | None | 3.4 |
| | [Homo sapiens] | | | |
| Rab33b | RAB33B, member of RAS oncogene family | NM 016858 | Golgi stack GTP-binding | 3.4 |
| | | | Lipoprotein Prenylation | |
| | | | Protein transport | |
| 9430065L19Rik | RIKEN cDNA 9430065L19 gene | NM 146083 | None | 3.4 |
| Pgr | progesterone receptor | NM 008829 | DNA-binding Nuclear | 3.4 |
| | | | protein Receptor Steroid- | |
| | | | binding Transcription | |
| | | | regulation Zinc-finger | |
| LOC218490 | similar to Transcription factor BTF3 (RNA | NM 145455 | Alternative splicing | 3.4 |
| | polymerase B transcription factor 3) | | Nuclear protein | |
| | | | Transcription regulation | |
| 4930434H03Rik | RIKEN cDNA 4930434H03 gene | none | None | 3.3 |
| Actn3 | Actinin alpha 3 | NM 013456 | Actin-binding Multigene | 3.3 |
| | | | family Repeat | |
| Mm.202311 | Mus musculus, clone IMAGE: 1379624, mRNA, | NA | GTP-binding Lipoprotein | 3.3 |
| | partial cds | | Membrane Multigene | |
| | | | family Palmitate | |
| | | | Transducer | |
| Gtpi | interferon-g induced GTPase | NM 019440 | None | 3.3 |
| Nat2 | N-acetyltransferase 2 (arylamine N- | NM 010874 | Acyltransferase Multigene | 3.3 |
| | acetyltransferase) | | family Polymorphism | |
| | | | Transferase | |
| Eya2 | eyes absent 2 homolog (Drosophila) | none | Alternative splicing | 3.3 |
| | | | Developmental protein | |
| | | | Multigene family | |
| 1110037N09Rik | RIKEN cDNA 1110037N09 gene | none | None | 3.2 |
| 5033414D02Rik | RIKEN cDNA 5033414D02 gene | NM 026362 | None | 3.1 |
| Mm.26147 | ESTs | NA | None | 3.1 |
| Il4 | interleukin 4 | NM 021283 | B-cell activation Cytokine | 3.1 |
| | | | Glycoprotein Growth | |
| | | | factor Signal | |
| Ubap1 | ubiquitin-associated protein 1 | NM 023305 | None | 3.1 |
| Acox1 | acyl-Coenzyme A oxidase 1, palmitoyl | NM 015729 | FAD Fatty acid | 2.9 |
| | | | metabolism Flavoprotein | |
| | | | Oxidoreductase | |
| | | | Peroxisome | |
| Ccl5 | chemokine (C-C motif) ligand 5 | NM 013653 | Chemotaxis Cytokine | 2.9 |
| | | | Inflammatory response | |
| | | | Signal T-cell | |
| AW457192 | expressed sequence AW457192 | NM 134084 | Cyclosporin Isomerase | 2.9 |
| | | | Mitochondrion Multigene | |
| | | | family Rotamase Transit | |
| | | | peptide | |
| 2610016K11Rik | RIKEN cDNA 2610016K11 gene | none | None | 2.8 |
| Fzd4 | frizzled homolog 4 (Drosophila) | NM 008055 | Developmental protein | 2.8 |
| | | | G-protein coupled receptor | |
| | | | Glycoprotein Multigene | |
| | | | family Signal | |
| | | | Transmembrane | |
| Pla2g4a | phospholipase A2, group IVA (cytosolic, | NM 008869 | Calcium Hydrolase Lipid | 2.8 |
| | calcium-dependent) | | degradation | |
| | | | Phosphorylation | |
| Scin | scinderin | NM 009132 | None | 2.7 |
| NA | AV239653 Mus musculus cDNA, 3 | AV239653 | None | 2.7 |
| | end/clone = 4732435F04 | | | |
| | /clone_end = 3/gb = AV239653 | | | |
| | /gi = 6192160/ug = Mm.88313 | | | |
| | /len = 214/NOTE = replacement for | | | |
| | probe set(s) 96411_f_at on MG-U74A mRNA | | | |
| Tcf12 | transcription factor 12 | NM 011544 | Alternative splicing | 2.7 |
| | | | Developmental protein | |
| | | | DNA-binding Nuclear | |
| | | | protein Transcription | |
| | | | regulation | |
| Madh7 | MAD homolog 7 (Drosophila) | NM 008543 | Alternative splicing | 2.7 |
| | | | Multigene family | |
| | | | Transcription regulation | |
| Gem | GTP binding protein (gene | NM 010276 | GTP-binding Membrane | 2.7 |
| | overexpressed in skeletal muscle) | | Phosphorylation | |
| Tpm1 | tropomyosin 1, alpha | NM 024427 | 3D-structure Acetylation | 2.7 |
| | | | Alternative splicing Coiled | |
| | | | coil Multigene family | |
| | | | Muscle protein | |
| | | | Phosphorylation Repeat | |
| Map17 | membrane-associated protein 17 | NM 026018 | None | 2.7 |
| Dcx | doublecortin | NM 010025 | Neurogenesis Neurone | 2.7 |
| | | | Phosphorylation Repeat | |
| Igk-V28 | immunoglobulin kappa chain variable 28 (V28) | none | Immunoglobulin C region | 2.6 |
| | | | Immunoglobulin domain | |
| Rnf11 | ring finger protein 11 | NM 013876 | None | 2.6 |
| Nfix | nuclear factor I/X | NM 010906 | None | 2.6 |
| Lin7c | lin 7 homolog c (C. elegans) | NM 011699 | None | 2.5 |
| Cln3 | ceroid lipofuscinosis, neuronal 3, juvenile | NM 009907 | Glycoprotein Lysosome | 2.5 |
| | (Batten, Spielmeyer-Vogt disease) | | Transmembrane | |
| Hhex | hematopoietically expressed homeobox | NM 008245 | Developmental protein | 2.5 |
| | | | DNA-binding Homeobox | |
| | | | Nuclear protein | |
| Gab1 | growth factor receptor bound protein | NM 021356 | None | 2.5 |
| | 2-associated protein 1 | | | |
| None | none | none | None | 2.5 |
| Kcnj3 | potassium inwardly-rectifying channel, | NM 008426 | Ion transport Ionic channel | 2.5 |
| | subfamily J, member 3 | | Potassium transport | |
| | | | Transmembrane Voltage- | |
| | | | gated channel | |
| Cradd | CASP2 and RIPK1 domain containing adaptor | NM 009950 | Apoptosis | 2.5 |
| | with death domain | | | |
| Mm.29914 | ESTs | NA | None | 2.4 |
| Fos | FBJ osteosarcoma oncogene | NM 010234 | DNA-binding Nuclear | 2.4 |
| | | | protein Phosphorylation | |
| | | | Proto-oncogene | |
| Mm.24247 | ESTs | NA | None | 2.4 |
| 4930472G13Rik | RIKEN cDNA 4930472G13 gene | NM 029447 | None | 2.4 |
| Ormdl3 | ORM1-like 3 (S. cerevisiae) | NM 025661 | None | 2.4 |
| Umpk | uridine monophosphate kinase | none | Kinase Transferase | 2.4 |
| Creg | cellular repressor of E1A-stimulated genes | NM 011804 | None | 2.4 |
| Utrn | utrophin | none | None | 2.3 |
| Mm.27769 | ESTs, Weakly similar to RIKEN cDNA 0610011E17 | NA | None | 2.3 |
| | [Mus musculus] [M. musculus] | | | |
| Igtp | interferon gamma induced GTPase | NM 018738 | None | 2.3 |
| Arg2 | arginase type II | NM 009705 | Arginine metabolism | 2.3 |
| | | | Hydrolase Manganese | |
| | | | Mitochondrion Transit | |
| | | | peptide Urea cycle | |
| Pklr | pyruvate kinase liver and red blood | NM 013631 | Alternative splicing | 2.2 |
| | cell | | Glycolysis Kinase | |
| | | | Magnesium Multigene | |
| | | | family Phosphorylation | |
| | | | Transferase | |
| 1810010A06Rik | RIKEN cDNA 1810010A06 gene | NM 026921 | None | 2.2 |
| Mm.532 | ESTs, Weakly similar to lysophospholipase 1; | NA | None | 2.2 |
| | phospholipase 1a; lysophopholipase 1 | | | |
| | [Mus musculus] [M. musculus] | | | |
| Vamp5 | vesicle-associated membrane protein 5 | NM 016872 | Multigene family | 2.2 |
| | | | Myogenesis Signal-anchor | |
| | | | Transmembrane | |
| 0710001O03Rik | RIKEN cDNA 0710001O03 gene | NM 146094 | None | 2.2 |
| 2610003J05Rik | RIKEN cDNA 2610003J05 gene | none | None | 2.2 |
| Tde1l | tumor differentially expressed 1, like | NM 019760 | None | 2.2 |
| Serpinf1 | serine (or cysteine) proteinase inhibitor, | NM 011340 | Glycoprotein Serpin Signal | 2.1 |
| | clade F), member 1 | | | |
| Scotin | scotin gene | NM 025858 | None | 2.1 |
| G3bp2 | Ras-GTPase-activating protein (GAP<120>) | NM 011816 | None | 2.1 |
| | SH3-domain binding protein 2 | | | |
| 1190002H23Rik | RIKEN cDNA 1190002H23 gene | NM 025427 | None | 2.1 |
| Nsccn1 | non-selective cation channel 1 | NM 010940 | None | 2.1 |
| Tgoln2 | trans-golgi network protein 2 | NM 009444 | None | 2.1 |
| Ywhae | tyrosine 3-monooxygenase/tryptophan | NM 009536 | None | 2.1 |
| | 5-monooxygenase activation protein, | | | |
| | epsilon polypeptide | | | |
| 4631408O11Rik | RIKEN cDNA 4631408O11 gene | none | None | 2.1 |
| Pou2af1 | POU domain, class 2, associating factor 1 | NM 011136 | Nuclear protein | 2.1 |
| | | | Transcription regulation | |
| Mm.220953 | Mus musculus, clone IMAGE: 4206769, | NA | None | 2.1 |
| | mRNA | | | |
| Casp6 | caspase 6 | NM 009811 | Apoptosis Hydrolase Thiol | 2.0 |
| | | | protease Zymogen | |
| None | none | none | Glycoprotein | 2.0 |
| | | | Immunoglobulin C region | |
| | | | Immunoglobulin domain | |
| Nr4a1 | nuclear receptor subfamily 4, group A, | NM 010444 | DNA-binding Nuclear | 2.0 |
| | member 1 | | protein Phosphorylation | |
| | | | Receptor Transcription | |
| | | | regulation Zinc-finger | |
| 1700023O11Rik | RIKEN cDNA 1700023O11 gene | NM 029339 | None | 2.0 |
| Brca2 | breast cancer 2 | NM 009765 | Polymorphism Repeat | 2.0 |
| H2-T22 | histocompatibility 2, T region locus 22 | NM 010397 | None | 2.0 |

TABLE 4
Genes With Upregulated Expression and Correlated Stem Cell Activity

| Symbol or | Gene Description or similarity | Corrrelation | |
|---|---|---|---|
| Acc. No. | to known proteins | to stem cell | Unigene No. |
| Rnps1 | ribonucleic acid binding | 1.000 | Mm.1951 |
| | protein S1 | | |
| Junb | Jun-B oncogene | 1.000 | Mm.1167 |
| Hdac3 | histone deacetylase 3 | 1.000 | Mm.20521 |
| Irf6 | interferon regulatory factor 6 | 1.000 | Mm.4179 |
| Gata3 | GATA binding protein 3 | 0.997 | Mm.606 |
| Xbp1 | X-box binding protein 1 | 0.993 | Mm.22718 |
| Cited2 | Cbp/p300-interacting | 0.992 | Mm.9524 |
| | transactivator, with Glu/Asp- | | |
| | rich carboxy-terminal domain, 2 | | |
| Nmyc1 | neuroblastoma myc-related | 0.986 | Mm.16469 |
| | oncogene I | | |
| Zfp292 | zinc finger protein 292 | 0.975 | Mm.38193 |
| Bdkrb1 | bradykinin receptor, beta 1 | 1.000 | Mm.57076 |
| Map17 | membrane-associated protein 17 | 0.995 | Mm.30181 |
| Ormdl3 | ORM1-like 3 (S. cerevisiae) | 0.990 | Mm.180546 |
| Fzd4 | frizzled homolog 4 | 0.988 | Mm.68712 |
| | (Drosophila) | | |
| Lgi4 | leucine-rich repeat LGI | 0.961 | Mm.1662 |
| | family, member 4 | | |
| Bdkrb1* | bradykinin receptor, beta 1 | 1.000 | Mm.57076 |
| Socs2 | suppressor of cytokine | 0.996 | Mm.4132 |
| | signaling 2 | | |
| Fzd4* | frizzled homolog 4 | 0.988 | Mm.68712 |
| | (Drosophila) | | |
| Kit* | kit oncogene | 0.961 | Mm.4394 |
| Inpp5d | inositol polyphosphate-5- | 0.958 | Mm.15105 |
| | phosphatase D | | |
| Fbxo9 | f-box only protein 9 | 1.000 | Mm.28584 |
| Nedd4 | neural precursor cell | 0.993 | Mm.16553 |
| | expressed, developmentally | | |
| | down-regulted gene 4 | | |
| Rnfl 1 | ring finger protein 11 | 0.992 | Mm.25228 |
| Ian 1 | immune associated nucleotide 1 | 0.999 | Mm.28395 |
| Iigp | interferon-inducible GTPase | 0.997 | Mm.29008 |
| Ifi47 | interferon gamma inducible | 0.984 | Mm.24769 |
| | protein | | |
| Tgtp | T-cell specific GTPase | 0.994 | Mm.15793 |
| Igtp | interferon gamma induced GTPase | 0.993 | Mm.858 |
| Gtpi | interferon-g induced GTPase | 0.989 | Mm.33902 |
| Serpinb6a | serine (or cysteine) proteinase | 0.996 | Mm.2623 |
| | inhibitor, clade B, member 6a | | |
| Serpina3g | serine (or cysteine) proteinase | 0.987 | Mm.15085 |
| | inhibitor, clade A, member 3G | | |
| Camk2b | calcium/calmodulin-dependent | 0.999 | Mm.4857 |
| | protein kinase II, beta | | |
| Gab1 | growth factor receptor bound | 0.997 | Mm.24573 |
| | protein 2-associated protein 1 | | |
| Gabarapl1 | gamma-aminobutyric acid | 0.997 | Mm.14638 |
| | (GABA(A)) receptor- | | |
| | associated protein-like 1 | | |
| Mtmr13 | myotubularin related protein 13 | 0.996 | Mm.200250 |
| Mt2 | metallothionein 2 | 0.999 | Mm.147226 |
| Car2 | carbonic anhydrase 2 | 0.995 | Mm.1186 |
| Cdkn 1c | cyclin-dependent kinase | 0.986 | Mm.168789 |
| | inhibitor 1C (P57) | | |
| Lcn7 | lipocalin 7 | 0.999 | Mm.15801 |
| A430017F18 | No similar gene | 1.000 | Mm.44883 |
| AU044919 | No significant similar gene | 1.000 | Mm.14438 |
| 2310075M17Rik | Similar to S3543 GTP-binding | 0.999 | Mm.196592 |
| | protein (90%) | | |
| E112 | Eleven-nineteen lysine-rich | 0.998 | Mm.21288 |
| | leukemia gene 2 | | |
| LOC207685 | Hypothetical protein | 0.998 | Mm.38214 |
| 2310061104Rik | No similar gene | 0.998 | Mm.5624 |
| 5830431A10Rik | Contain Corl/Xlr/Xmr | 0.997 | Mm.1148 |
| | conserved region | | |
| 2700007P21Rik | Unknown protein | 0.997 | Mm.3587 |
| B930086G17 | No similar gene | 0.992 | Mm.24738 |
| 2410166105Rik | Hypothetical protein | 0.989 | Mm.30153 |
| D10Ertd749e | Similar to ZW10 interacting | 0.986 | Mm.38994 |
| | protein-1 | | |
| 2210023F24Rik | Contain B-box Zn-finger and | 0.983 | Mm.5510 |
| | SPRY domain | | |
| Riken 4237666 | No significant similar gene | 0.978 | Mm.276231 |
| 6230421P05Rik | No similar gene | 0.978 | Mm.26147 |
| 4631408O11Rik* | No significant similar gene | 0.964 | Mm.2935 |
| 1110054N06Rik* | Unknow protein with Ankyrin | 0.960 | Mm.15351 |
| | repeat | | |

TABLE 5
Genes down-regulated in CD38+CD34− Cells

| Symbol or | | Correlation | Unigene |
|---|---|---|---|
| Acc. No. | Description | to SC activity | No. |
| Satb1 | Special AT-rich sequence binding protein 1 | 0.955 | Mm.4381 |
| Ptpro | Protein tyrosine phosphatase, receptor type, O | 0.999 | Mm.4715 |
| Sell | Selectin, lymphocyte | 0.988 | Mm.1461 |
| Ccl9 | Chemokine (C-C motif) ligand 9 | 0.988 | Mm.2271 |
| Cnn3 | Calponin 3, acidic | 0.988 | Mm.22171 |
| Lgals3 | Lectin, galactose binding, soluble 3 | 0.971 | Mm.2970 |
| Mki67 | Antigen identified by monoclonal antibody Ki 67 | 0.998 | Mm.4078 |
| Bin1 | Bridging integrator 1 | 0.977 | Mm.4383 |
| Sult4a1 | Sulfotransferase family 4A, member 1 | 1.000 | Mm.20451 |
| Hdc | Histidine decarboxylase | 0.996 | Mm.18603 |
| AI132321 | Contain phospholipase D. active site motif | −1.000 | Mm.203915 |
| 2610036L13Rik | No similar gene | −1.000 | Mm.23526 |
| BC018347 | Similar to translation initiation factor 1F-2 | −1.000 | Mm.154309 |
| X90778 | Similar to Histone H2B | −1.000 | Mm.21579 |
| AW060549 | Similar to Retrovirus-related POL polyprotein | −0.999 | Mm.29177 |
| X67863 | Similar to Octapeptide-repeat protein T2 | −0.995 | Mm.35868 |
| X15378 | Similar to Myeloperoxidase and Eosinophil | −0.975 | Mm.4668 |
| | peroxidase precursor | | |
| Plac8 | Uncharacterized Cys-rich domain containing protein | −0.960 | Mm.34609 |
| D13Ertd275e | Hypothetical protein | −0.952 | Mm.21231 |

TABLE 6
Cassification and Characterization of Genes Upregulated in Mouse HSCs

| | | | Sequence | Unigene | Protein |
|---|---|---|---|---|---|
| Class | Name | Sequence Description | Code | Code | ID |
| Apoptosis | Birc5 | baculoviral IAP repeat-containing 5 | 101521 | Mm.8552 | O70201 |
| Cell cycle | Spin | spindlin | 99563 | Mm.42193 | |
| Chromosomal | Btg1 | M. musculus btg1 mRNA. | 93104 | | P31607 |
| Chromosomal | Calm2 | Mus musculus calmodulin synthesis (CaM) | 93293 | | P02593 |
| | | cDNA, complete cds. | | | |
| Enzyme | Ctsl | cathepsin L | 101963 | Mm.930 | P06797 |
| Enzyme | Gdi1 | guanosine diphosphate (GDP) dissociation | 97313 | Mm.205830 | P50396 |
| | | inhibitor 1 | | | |
| Enzyme | Hadh2 | hydroxysteroid (17-beta) dehydrogenase 10 | 101045 | Mm.6994 | O08756 |
| Enzyme | Mt2 | Mouse metallothionein II (MT-II) gene. | 101561 | | P02798 |
| Enzyme | Pnp | purine-nucleoside phosphorylase | 93290 | Mm.17932 | P23492 |
| Enzyme | Vdu1 | Vhlh-interacting deubiquitinating enzyme 1 | 160710 | Mm.24383 | |
| Kinase | Csnk1e | casein kinase 1, epsilon | 97925 | Mm.30199 | Q9QUI3 |
| Kinase | Nme3 | expressed in non-metastatic cells 3 | 94981_i | Mm.27278 | |
| Lectin | Lgals9 | lectin, galactose binding, soluble 9 | 103335 | Mm.18087 | O08573 |
| Metabolism | Aldh1a1 | aldehyde dehydrogenase family 1, | 100068 | Mm.4514 | P24549 |
| | | subfamily A1 | | | |
| Metabolism | Aldh1a7 | aldehyde dehydrogenase family 1, | 94778 | Mm.14609 | O35945 |
| | | subfamily A7 | | | |
| Metabolism | Cpo | coproporphyrinogen oxidase | 98505_i | Mm.35820 | P36552 |
| Metabolism | Cpo | coproporphyrinogen oxidase | 98506_r | Mm.35820 | P36552 |
| Metabolism | Ech1 | enoyl coenzyme A hydratase 1, peroxisomal | 93754 | Mm.2112 | O35459 |
| Metabolism | Mtcp1 | M. musculus MTCP-1 gene. | 103043 | | Q61908 |
| Nuclear | Rbmx | RNA binding motif protein, X chromosome | 97848 | Mm.28275 | Q9R0Y0 |
| Nuclear | Snrpa | small nuclear ribonucleoprotein polypeptide | 100101 | Mm.4633 | Q62189 |
| | | A | | | |
| Secreted | Iap | intracisternal A particles | 97181_f | Mm.212712 | P03975 |
| Secreted | Tff2 | Mus musculus spasmolytic polypeptide | 93302 | | Q03404 |
| | | (mSP) gene, complete cds. | | | |
| Signaling | Gnb4 | guanine nucleotide binding protein, beta 4 | 93949 | Mm.9336 | P29387 |
| Signaling | Tsc2 | tuberous sclerosis 2 | 97953_g | Mm.30435 | Q61037 |
| Structural | Fscn1 | fascin homolog 1, actin bundling protein | 92838 | Mm.13194 | Q61553 |
| | | (Strongylocentrotus) purpuratus) | | | |
| Transcription | Irf1 | Interferon regulatory factor 1 | 102401 | Mm.1246 | P15314 |
| Transcription | Cited2 | Cbp/p300-interacting transactivator, with | 101973 | Mm.9524 | O35740 |
| | | Glu/Asp-rich carboxy-terminal domain, 2 | | | |
| Transcription | Ncor1 | nuclear receptor co-repressor 1 | 101536 | Mm.88061 | Q60974 |
| Transcription | Sox6 | SRY-box containing gene 6 | 92726 | Mm.4656 | P40645 |
| Transcription | Hhex | Mus musculus Hex(Prh) gene, exon4 and | 98408 | Mm.33896 | Q9R1X2 |
| | | complete cds. | | | |
| Transcription | Trim30 | tripartite motif protein 30 | 98030 | Mm.3288 | P15533 |
| Transcription | Tieg | TGFB inducible early growth response | 99602 | Mm.4292 | O89091 |
| Transcription | Klf2 | Kruppel-like factor 2 (lung) | 96109 | Mm.26938 | Q60843 |
| Transcription | Eif4a2 | eukaryotic translation initiation factor 4A2 | 93089 | Mm.16323 | P10630 |
| Transcription | H2a-615 | Mus musculus histone H2a.2-615 (H2a- | 93068_r | | P20670 |
| | | 615), and histone H3.2-615 (H3-615) | | | |
| | | genes, complete cds. | | | |
| Transcription | Nfe212 | Mus musculus p45 NF-E2 related factor 2 | 92562 | | Q60795 |
| | | (NRF2) gene, exon 2 to exon 5 and complete cds. | | | |
| Transcription | Fli1 | Friend leukemia integration 1 | 94698 | Mm.119781 | P26323 |
| Transcription | Mcmd5 | mini chromosome maintenance deficient 5 | 100156 | Mm.5048 | P49718 |
| | | (S. cerevisiae) | | | |
| Transcription | H3f3b | H3 histone, family 3B | 100708 | Mm.18516 | P06351 |
| Transcription | Rev3l | REV3-like, catalytic subunit of DNA | 103457 | Mm.2167 | Q61493 |
| | | polymerase zeta RAD54 like (S. cerevisiae) | | | |
| Transcription | Hoxb5 | homeo box B5 | 103666 | Mm.207 | P09079 |
| Transcription | Pbx1 | pre B-cell leukemia transcription factor 1 | 94804 | Mm.221246 | P41778 |
| Transcription | Zfp3611 | zinc finger protein 36, C3H type-like 1 | 93324 | Mm.18571 | P23950 |
| Transcription | Myb | myeloblastosis oncogene | 92644_s | Mm.1202 | P06876 |
| Transcription | Sp4 | trans-acting transcription factor 4 | 92992_i | Mm.5073 | |
| Transcription | Idb2 | Mus musculus helix-loop-helix protein Id2 | 93013 | | |
| | | gene, 3′; region. | | | |
| Transmembrane | Hiat1 | hippocampus abundant gene transcript 1 | 160447 | Mm.3792 | P70187 |
| Transmembrane | Igh-4 | mouse gene for the constant part of gamma- | 101870 | | P01869 |
| | | 1 immunogloblin. | | | |
| Transmembrane | Ii | Ia-associated invariant chain | 101054 | Mm.7043 | P04441 |
| Transmembrane | H2-Aa | histocompatibility 2, class II antigen A, | 92866 | Mm.175310 | P23150 |
| | | alpha | | | |
| Transmembrane | Epor | Mouse gene for erythropoietin receptor. | 103997 | | P14753 |
| Transmembrane | Irs2 | Mus musculus insulin receptor substrate-2 | 92205 | | O88970 |
| | | (Irs2) gene, partial cds. | | | |
| Transmembrane | H2-Eb1 | histocompatibility 2, class 11 antigen E beta | 94285 | Mm.22564 | Q61857 |
| Transmembrane | Tnfrsf17 | tumor necrosis factor receptor superfamily, | 94190 | Mm.12935 | O88472 |
| | | member 17 | | | |
| Transmembrane | Adcy9 | adenylate cyclase 9 | 92527 | Mm.4294 | P51830 |
| Transmembrane | Edg1 | endothelial differentiation sphingolipid G- | 161788_f | Mm.982 | |
| | | protein-coupled receptor 1 | | | |
| Transmembrane | Fzd4 | frizzled homolog 4 (Drosophila) | 93459_s | Mm.68712 | |
| Transport | Vps35 | vacuolar protein sorting 35 | 92640 | Mm.196201 | Q9EQH3 |
| Transport | Hbb-b2 | Mouse gene for beta-1-globin. | 103534 | | P02089 |
| Transport | Kpnb1 | karyopherin (importin) beta 1 | 93111 | Mm.16710 | P70168 |
| Transport | Rab9 | RAB9, member RAS oncogene family | 95516 | Mm.25306 | Q9R0M6 |
| Transport | Rac1 | RAS-related C3 botulinum substrate 1 | 101555 | Mm.889 | P15154 |
| Transport | Rab33b | Mus musculus DNA for Rab33B, exon 2 | 103062 | | O35963 |
| | | and complete cds. | | | |
| Zinc Finger | Zfp216 | zinc finger protein 216 | 160321 | Mm.2904 | O88878 |
| Zinc Finger | Rnf11 | ring finger protein 11 | 160205_f | Mm.25228 | Q9QYK7 |
| Zinc Finger | Nbr1 | next to the Brca1 | 101484 | Mm.784 | P97432 |
| Zinc Finger | pol | Mus musculus clone MIA14 full-length | 93907_f | | P11365 |
| | | intracisternal A-particle gag protein gene, | | | |
| | | complete cds; and pol pseudogene, | | | |
| | | complete sequence. | | | |
| Zinc Finger | Gfi1b | growth factor independent 1B | 102260 | Mm.10804 | O70237 |
| Zinc Finger | Car1 | carbonic anhydrase 1 | 98098 | Mm.3471 | P13634 |
| | Cul4a | cullin 4A | 104288 | Mm.22276 | |
| | D7Wsu128e | DNA segment, Chr 7, Wayne State | 103861_s | Mm.21103 | |
| | | University 128, expressed | | | |
| | Rhced | Rhesus blood group CE and D | 103340 | Mm.195461 | Q9QX04 |
| | AU044919 | expressed sequence AU044919 | 102823 | Mm.14438 | |
| | Igj | immunoglobulin joining chain | 102372 | Mm.1192 | |
| | Lisch7 | liver-specific bHLH-Zip transcription factor | 162274_f | Mm.4067 | |
| | Igh-VJ558 | immunoglobulin heavy chain, (J558 family) | 161486_f | Mm.157783 | |
| | 0910001L24 | RIKEN cDNA 0910001L24 gene | 161243_f | Mm.22637 | |
| | Rik | | | | |
| | Txnip | thioredoxin interacting protein | 160547_s | Mm.77432 | |
| | Dr1 | down-regulator of transcription 1 | 160449 | Mm.38184 | |
| | 4933429H19 | Mus musculus, Similar to translocation | 160136_r | Mm.200980 | |
| | Rik | protein 1, clone IMAGE: 5347105, mRNA, | | | |
| | | partial cds | | | |
| | 1500010B24 | RIKEN cDNA 1500010B24 gene | 160111 | Mm.65264 | |
| | Rik | | | | |
| | IgM | Mus castaneus IgK chain gene, C-region, 3′; end. | 102156_f | | |
| | AA409749 | expressed sequence AA409749 | 100742 | Mm.3628 | |
| | D2Ertd63e | DNA segment, Chr 2, ERATO Doi 63, | 95862 | Mm.24965 | |
| | | expressed | | | |
| | Igk-V28 | Mus musculus anti-HIV-1 reverse | 100322 | Mm.220154 | |
| | | transcriptase single-chain variable fragment | | | |
| | | mRNA, complete cds | | | |
| | 5830431A10 | RIKEN cDNA 5830431A10 gene | 94136 | Mm.1148 | |
| | Rik | | | | |
| | Igl-V1 | Mouse Ig active lambda-1-chain C-region | 93638_s | | |
| | | gene, 3′; end. | | | |
| | Imap38 | immunity-associated protein, 38 kDa | 92489 | Mm.197478 | P70224 |
| | 92316_f | Mouse germline Ig lambda-2-chain C- | 92316_f | | |
| | | region gene, 3′; end. | | | |
| | 2700007P21 | RIKEN cDNA 2700007P21 gene | 92268 | Mm.3587 | |
| | Rik | | | | |
| | 104477 | ESTs | 104477 | Mm.29940 | |
| | 0610012A05 | RIKEN cDNA 0610012A05 gene | 104206 | Mm.27619 | |
| | Rik | | | | |
| | Atp6s1 | Mus musculus, clone MGC: 37615 | 103699_i | Mm.222723 | |
| | | IMAGE: 4989784, mRNA, complete cds | | | |
| | Gbp3 | guanylate nucleotide binding protein 3 | 103202 | Mm.1909 | |
| | immunoglobulin | Mouse mRNA for immunoglobulin gamma- | 102721 | | |
| | V region | 3 V-D-J region and secreted constant | | | |
| | | region, complete cds. | | | |
| | AI256744 | Mus musculus, clone IMAGE: 3500612, | 102233 | Mm.1043 | |
| | | mRNA, partial cds | | | |
| | Ptdss1 | phosphatidylserine synthase 1 | 101931 | Mm.9440 | O55024 |
| | Gga1 | golgi associated, gamma adaptin ear | 98445 | Mm.34525 | |
| | | containing, ARF binding protein 1 | | | |
| | 4121402D02 | RIKEN cDNA 4121402D02 gene | 97935 | Mm.30252 | |
| | Rik | | | | |
| | Iigp | interferon-inducible GTPase | 96764 | Mm.29008 | Q9Z1M3 |
| | 2310022K15 | RIKEN cDNA 2310022K15 gene | 95622 | Mm.28047 | |
| | Rik | | | | |
| | Vcl | vinculin | 94963 | Mm.12842 | |
| | 2610319K07 | RIKEN cDNA 2610319K07 gene | 104744 | Mm.200479 | |
| | Rik | | | | |
| | Iga | Mouse Ig germline D-J-C region alpha gene | 100583 | | |
| | | and secreted tail; Mouse germ line gene for | | | |
| | | immunoglobulin alpha H constant part | | | |
| | | (coding for the last three exons) | | | |
| | Prpf8 | pre-mRNA processing factor 8 | 98574 | Mm.3757 | |
| | Scotin | scotin gene | 95102 | Mm.196533 | |
| | 1110035L05 | RIKEN cDNA 1110035L05 gene | 95052 | Mm.29140 | |
| | Rik | | | | |
| | 3110001A13 | RIKEN cDNA 3110001A13 gene | 96640 | Mm.200627 | |
| | Rik | | | | |
| | Vps26 | vacuolar protein sorting 26 (yeast) | 96665 | Mm.27373 | |
| | mu- | Mouse germ line gene fragment for mu- | 93583_s | | |
| | immunoglobulin | immunoglobulin C-terminus (secreted | | | |
| | | form). | | | |
| | H19 | M. musculus H19 mRNA. | 93028 | | Q61638 |
| | Car2 | carbonic anhydrase 2 | 92642 | Mm.1186 | |
| | Rae1 | RAE1 RNA export 1 homolog (S. pombe) | 160466 | Mm.4113 | |
| | Map1lc3 | microtubule-associated protein 1 light chain 3 | 160288 | Mm.28357 | |
| | 1700008C22 | RIKEN cDNA 1700008C22 gene | 160123 | Mm.177990 | |
| | Rik | | | | |
| | 98254_f | un98f06.x1 NCI_CGAP_Mam6 Mus musculus | 98254_f | | |
| | | cDNA clone IMAGE: 2581955 3′; | | | |
| | | similar to gb: M10062 Mouse IgE-binding | | | |
| | | factor mRNA, complete cds (MOUSE); | | | |
| | | mRNA sequence. | | | |
| | Eef2 | eukaryotic translation elongation factor 2 | 97559 | Mm.27818 | Q61509 |
| | Igk-V28 | immunoglobulin kappa chain variable 28 | 99405 | Mm.104747 | |
| | | (V28) | | | |
| | 9030022E12 | RIKEN cDNA 9030022E12 gene | 104198 | Mm.27519 | |
| | Rik | | | | |
| | D18362 | expressed sequence D18362 | 103206 | Mm.205433 | |
| | Hey1 | Mus musculus 6 days neonate head cDNA, | 101913 | Mm.222825 | |
| | | RIKEN full-length enriched library, | | | |
| | | clone: 5430408K11: hairy/enhancer-of-split | | | |
| | | related with YRPW motif 1, full insert | | | |
| | | sequence | | | |
| | shrm | shroom | 100024 | Mm.46014 | |
| | AW547365 | expressed sequence AW547365 | 97425 | Mm.30015 | |
| | D8Ertd69e | DNA segment, Chr 8, ERATO Doi 69, | 94922_i | Mm.26609 | |
| | | expressed | | | |
| | Frap1 | FK506 binding protein 12-rapamycin | 104708 | Mm.21158 | |
| | | associated protein 1 | | | |
| | 4933434E20 | RIKEN cDNA 4933434E20 gene | 104038 | Mm.21451 | |
| | Rik | | | | |
| | 1810009A16 | RIKEN cDNA 1810009A16 gene | 104041 | Mm.21458 | |
| | Rik | | | | |
| | Pex11a | peroxisomal biogenesis factor 11a | 103660 | Mm.20615 | Q9Z211 |
| | AU044919 | expressed sequence AU044919 | 102824_g | Mm.14438 | |
| | MGC29044 | hypothetical protein MGC29044 | 102375 | Mm.1196 | |
| | Mkm1 | makorin, ring finger protein, 1 | 101070 | Mm.7198 | |
| | LOC207933 | similar to Isopentenyl-diphosphate delta- | 96269 | Mm.29847 | |
| | | isomerase (IPP isomerase) (Isopentenyl | | | |
| | | pyrophosphate isomerase) | | | |
| | Elp3 | elongation protein 3 homolog (S. cerevisiae) | 95717 | Mm.29719 | |
| | Add1 | adducin 1 (alpha) | 94535 | Mm.29052 | |
| | Pbef | pre-B-cell colony-enhancing factor | 94461 | Mm.28830 | |
| | 4930588A18 | Mus musculus, clone IMAGE: 4457493, mRNA | 96717 | Mm.233830 | |
| | Rik | | | | |
| | Dad1 | Mus musculus Defender against Apoptotic | 96008 | | |
| | | Death (Dad1) gene, exon 3. | | | |
| | 2410015A15 | RIKEN cDNA 2410015A15 gene | 95433 | Mm.24495 | |
| | Rik | | | | |
| | Xbp1 | X-box binding protein 1 | 94821 | Mm.22718 | |
| | Net1 | neuroepithelial cell transforming gene 1 | 94223 | Mm.22261 | Q9Z1L7 |
| | Igk-V28 | immunoglobulin kappa chain variable 28 | 93086 | Mm.104747 | |
| | | (V28) | | | |
| | LOC218490 | similar to Transcription factor BTF3 (RNA | 93057 | Mm.1538 | |
| | | polymerase B transcription factor 3) | | | |
| | Lamc1 | laminin, gamma 1 | 161706_f | Mm.1249 | |
| | AI450287 | expressed sequence AI450287 | 161596_f | Mm.222827 | |
| | Sep15 | 15-kDa selenoprotein | 160360 | Mm.29812 | |
| | LOC229906 | similar to TRANSCRIPTION INITIATION | 160225 | Mm.27213 | |
| | | FACTOR IIB (TFIIB) (RNA | | | |
| | | POLYMERASE II ALPHA INITIATION | | | |
| | | FACTOR) | | | |
| | 2810043O03 | RIKEN cDNA 2810043O03 gene | 98756 | Mm.45532 | |
| | Rik | | | | |
| | 96532 | ESTs, Highly similar to nucleolar protein | 96532 | Mm.35019 | |
| | | GU2 [Mus musculus] [M. musculus] | | | |
| | Myt1l | myelin transcription factor 1-like | 96495 | Mm.2523 | P97500 |
| | 2010004A03 | RIKEN cDNA 2010004A03 gene | 94802 | Mm.35302 | |
| | Rik | | | | |
| | C79248 | expressed sequence C79248 | 94689 | Mm.153895 | |
| | Mylk | myosin, light polypeptide kinase | 93482 | Mm.27680 | |
| | D1Ertd147e | DNA segment, Chr 1, ERATO Doi 147, | 93191 | Mm.5572 | |
| | | expressed | | | |
| | R75364 | expressed sequence R75364 | 92397 | Mm.89393 | |
| | 92245 | ESTs, Highly similar to nucleolar protein | 92245 | Mm.35019 | |
| | | GU2 [Mus musculus] [M. musculus] | | | |
| | Ctse | Mus musculus cathepsin E gene, exon 1, | 104696 | | |
| | | partial. | | | |
| | AA420392 | expressed sequence AA420392 | 104670 | Mm.32357 | |
| | Acyp2 | acylphosphatase 2, muscle type | 104258 | Mm.28407 | |
| | Lrba | LPS-responsive beige-like anchor | 104264 | Mm.28458 | |
| | Dock2 | dedicator of cyto-kinesis 2 | 103462 | Mm.2173 | |
| | Gabpa | GA repeat binding protein, alpha | 103440 | Mm.18974 | |
| | Nrip1 | nuclear receptor interacting protein 1 | 103288 | Mm.20895 | Q9Z2K2 |
| | AI225904 | expressed sequence AI225904 | 103200 | Mm.1902 | |
| | 98438_f | Mouse Q4 class 1 MHC gene (exon 5). | 98438_f | | Q31220 |
| | 2010012D11 | RIKEN cDNA 2010012D11 gene | 96231 | Mm.140243 | |
| | Rik | | | | |
| | AU019574 | Mus musculus, Similar to hypothetical | 96172 | Mm.28395 | |
| | | protein FLJ11110, clone MGC: 11734 | | | |
| | | IMAGE: 3968418, mRNA, complete cds | | | |
| | 9130415E20 | RIKEN cDNA 9130415E20 gene | 95020 | Mm.40620 | |
| | Rik | | | | |
| | 95021 | Mus musculus, clone IMAGE: 4502890, | 95021 | Mm.27476 | |
| | | mRNA | | | |
| | AW495846 | expressed sequence AW495846 | 104549 | Mm.23702 | |
| | Gtpbp2 | GTP binding protein 2 | 104144 | Mm.22147 | |
| | 2310050N11 | RIKEN cDNA 2310050N11 gene | 104114 | Mm.21954 | |
| | Rik | | | | |
| | Ormd13 | ORM1-like 3 (S. cerevisiae) | 98065 | Mm.180546 | |
| | 2610003J05 | RIKEN cDNA 2610003J05 gene | 97491 | Mm.31051 | |
| | Rik | | | | |
| | Map17 | membrane-associated protein 17 | 96935 | Mm.30181 | |
| | Gabarapl2 | GABA(A) receptor-associated protein like 2 | 96840 | Mm.30017 | |
| | 2310050K10 | RIKEN cDNA 2310050K10 gene | 95743 | Mm.29769 | |
| | Rik | | | | |
| | AI182287 | expressed sequence AI182287 | 94469 | Mm.28848 | |
| | Nudel | nuclear distribution gene E-like | 98884_r | Mm.31979 | |
| | CpneI | copine I | 97199 | Mm.27660 | |
| | Dnajb9 | DnaJ (Hsp40) homolog, subfamily B, | 96680 | Mm.27432 | |
| | | member 9 | | | |
| | 95488 | Mus musculus, clone IMAGE: 3597827, | 95488 | Mm.25018 | |
| | | mRNA, partial cds | | | |
| | 2700059C12 | RIKEN cDNA 2700059C12 gene | 93312 | Mm.18485 | |
| | Rik | | | | |
| | Sdcbp | syndecan binding protein | 93017 | Mm.14744 | O88601 |

TABLE 7
Tansmembrane Proteins Enriched in Mouse HSCs

| Classification | Description |
|---|---|
| surface | Histocompatibility 2, class II antigen |
| antigen | E beta |
| receptor | Gamma-aminobutyric acid (GABA) B |
| | receptor, 1 |
| oncogene | Myeloproliferative leukemia virus |
| | oncogene (TPOR) |
| surface | Histocompatibility 2, class II antigen |
| antigen | A alpha |
| | Cytotoxic T lymphocyte-associated |
| | protein 2 beta |
| receptor | Erythropoietin receptor |
| oncogene | Kit oncogene |
| | Coagulation factor II (thrombin) |
| | receptor |
| | Frizzled homolog 4 (Drosophila) |
| | Membrane-associated protein 17 |
| surface | ESTs similar to C211_Human putative |
| glycoprotein | surface glycoprotein |

TABLE 8
Transcription Factors Upregulated in Mouse HSCs

| Symbol | Description | Fold change | Accession No. |
|---|---|---|---|
| Klf2 | Kruppel-like factor 2 (lung) | 44.9 | NM 008452 |
| Nmyc1 | neuroblastoma myc-related | 10.4 | NM 008709 |
| | oncogene 1 | | |
| Zfxlha | zinc finger homeobox 1 a | 10.4 | NM 011546 |
| Gata3 | GATA-binding protein 3 | 9.0 | NM 008091 |
| Tcfl5 | transcription factor 15 | 8.6 | NM 009328 |
| Tall | T-cell acute lymphocytic | 8.3 | NM 011527 |
| | leukemia 1 | | |
| Hoxb5 | homeo box B5 | 7.2 | NM 008268 |
| Meis1 | myeloid ecotropic viral | 7.1 | NM 010789 |
| | integration site 1 | | |
| Pbx3b | Mus musculus transcription | 6.5 | AF020200 |
| | factor PBX3b | | |
| Cited2 | Cbp/p300-interacting | 5.2 | NM 010828 |
| | transactivator 2 | | |
| Atf2 | activating transcription factor 2 | 3.6 | none |
| Pbx1 | pre B-cell leukemia | 4.7 | NM 008783 |
| | transcription factor 1 | | |
| None | chromatin remodeling factor | 4.5 | Mm.24637 |
| None | EST similar to PRE-MRNA | 3.4 | Mm.29915 |
| | SPLICING FACTOR SRP20 | | |
| Btf3 | basic transcription factor 3 | 3.2 | none |
| Tcfl2 | transcription factor 12 | 2.7 | NM 011544 |
| Madh7 | MAD homolog 7 (Drosophila) | 2.7 | NM 008543 |
| Hhex | hematopoietically expressed | 2.5 | NM 008245 |
| | homeobox | | |

### Example 5

### Hierarchical Clustering Analysis of Differential Expressed Genes

This Example describes study aimed at determining if genes differentially expressed with the HSC compartment are also expressed in other tissues. To perform this analysis we compared the gene expression levels of 210 differentially expressed HSC genes with a database composed of 45 normal tissue. Hierarchical clustering of these data was used to group both those tissues and genes with similar expression patterns. The three HSC cell subsets formed a distinct branch in this analysis, with LTR-enriched 38⁺34⁻ cells forming a discrete branch compared to the STR cells (38⁺34⁺ and 38⁻34⁺). This clustering pattern is consistent with the stem cell activity pattern within the three subsets. Importantly, the HSC samples do not cluster near the bone or bone marrow samples suggesting that the differentially expressed HSC genes are not bone marrow related. This analysis also showed that the majority of these genes were not ubiquitously expressed although most were expressed at comparable levels in at least one other tissue.

Three of the genes were found to have their peak expression within the HSC compartment. These were the scaffolding protein Gab1 (GRB2-asssociated binding protein 1) and the uncharacterized gene A430017F18 which displayed the highest level expression in the LTR enriched CD38⁺CD34⁻ cells, and the Pdgfrb gene (platelet derived growth factor receptor, beta polypeptide) which peaked within the 38⁺34⁺ STR HSC subset. Although the majority of these genes are also expressed at comparable levels in other tissues it is important to note that in many cases the level of expression in HSC subsets was at or near the peak expression determined for these genes across the entire 45 tissue panel. The high relative expression within HSCs of this subset of genes indicates that they likely to play an important role in the biology of HSCs.

It is understood that the examples and embodiments described herein are for illustrative purposes only and that various modifications or changes in light thereof will be suggested to persons skilled in the art and are to be included within the spirit and purview of this application and scope of the appended claims. Although any methods and materials similar or equivalent to those described herein can be used in the practice or testing of the present invention, the preferred methods and materials are described.

All publications, GenBank sequences, patents and patent applications cited herein are hereby expressly incorporated by reference in their entirety and for all purposes as if each is individually so denoted.

We claim:

1. A method for inhibiting differentiation of mammalian stem cells, comprising (a) providing a population of stem cells, (b) introducing a vector comprising an HSC differentiation-inhibiting polynucleotide sequence shown in Table 1 and Table 4 into the stem cells, and (c) expressing a polypeptide encoded by the polynucleotide by culturing the modified stem cells, thereby inhibiting differentiation of the stem cells.

2. The method of claim 1, wherein the population of stem cells are isolated from bone marrow.

3. The method of claim 1, wherein the stem cells are human hematopoietic stem cells.

4. The method of claim 3, wherein the stem cells are first selected for expression of CD34 and Thy prior to introducing the vector.

5. The method of claim 1, wherein the stem cells are mouse hematopoietic stem cells.

6. The method of claim 5, wherein the stem cells are first selected for expression of CD38 and lack of expression of CD34 prior to introducting the vector.

7. The method of claim 1, wherein the HSC differentiation-inhibiting polynucleotide encodes GATA-binding protein 3 (Gata3) or ID3.

8. A method for increasing the effective dose of hematopoietic stem cells in a mammalian subject, comprising (a) providing a population of hematopoietic stem cells, (b) introducing into the cells an HSC differentiation-inhibiting polynucleotide selected from Table 1 and Table 4, and (c) administering the genetically modified cells that express an HSC differentiation-inhibiting polypeptide to a mammalian subject; thereby increasing the effective dose of hematopoietic stem cells in the subject.

9. The method of claim 8, wherein the administered stem cells are a subpopulation of the modified cells that are selected for expression of the polypeptide prior to administering to the subject.

10. The method of claim 8, wherein the administered stem cells overexpress the HSC differentiation-inhibiting polypeptide.

11. The method of claim 8, wherein the hematopoietic stem cells are obtained from bone marrow.

12. The method of claim 8, wherein the subject is human, and the hematopoietic stem cells are human hematopoietic stem cells.

13. The method of claim 12, wherein the hematopoietic stem cells are selected for expression of CD38 and Thy prior to introduction of the HSC differentiation-inhibiting polynucleotide.

14. The method of claim 8, wherein an expression vector comprising the HSC differentiation-inhibiting polynucleotide is introduced into the cells.

15. A method for inhibiting hematopoietic stem cell differentiation, comprising contacting a population of HSCs with an effective amount of an HSC differentiation-inhibiting polypeptide selected from Tables 1 and 4, thereby inhibiting differentiation of the HSCs.

16. The method of claim 15, wherein the HSCs are present in an in vitro cell culture.

17. The method of claim 15, wherein the HSCs are present in a subject grafted with the HSCs.

18. The method of claim 15, wherein the subject is human, and the HSC differentiation-inhibiting polypeptide is selected from the group shown in Table 2.

19. A method for isolating a population of cells that are enriched for hematopoietic stem cells (HSCs), the method comprising (a) obtaining a sample of cells containing hematopoietic stem cells, (b) selecting cells from the sample based on expression or lack of expression of at least one known HSC surface marker, and at least one molecule shown in Table 2 and Table 7 and (c) separating cells with the known HSC marker and at least one of the molecules shown in Table 2 and Table 7 thereby isolating a population of human cells enriched for hematopoietic stem cells.

20. The method of claim 19, wherein the hematopoietic stem cells are human HSCs.

21. The method of claim 20, wherein the known HSC marker is CD34⁺ and Thy⁺.

22. The method of claim 20, wherein the at least one molecule is a surface molecule shown in Table 2.

23. The method of claim 19, wherein the hematopoietic stem cells are mouse HSCs.

24. The method of claim 23, wherein the known HSC marker is CD38⁺ and CD34⁻.

25. The method of claim 23, wherein the isolated population of cells are also selected for expression of c-kit and Sca-1 but lack of expression of Lin.

26. The method of claim 19, wherein the sample of cells are obtained from bone marrow.

27. A method of enumerating hematopoietic stem cells in a population of cells, comprising (a) contacting the population of cells with an antibody that specifically binds to one HSC surface marker shown in Table 2 and Table 7 under conditions which allow the antibody to specifically bind to the HSC surface marker; and (b) quantifying the cells recognized by the antibody; thereby enumerating hematopoietic stem cells in the population of cells.

28. The method of claim 27, wherein the population of cells is a mixture of hematopoietic cells.

29. The method of claim 27, wherein hematopoietic stem cells are human HSCs, and the population of cells are first selected for expression of CD34 and Thy prior to the contacting.

30. The method of claim 27, wherein hematopoietic stem cells are mouse HSCs, and the population of cells are first selected for expression of CD38 but lack of expression of CD34 prior to the contacting.
